SEQUENCE LISTING (132 sequences)

SEQ ID NO: 1 - protein, 1594 aa, Unknown [sequence omitted]

SEQ ID NO: 2 - protein, 1588 aa, Unknown
  mouse Rb1cc1
MKLYVFLVNTGTTLTFDTELTVQTVADLKHAIQSKYKIAIQHQVLVVNGGECMAADRRVCTYSAGTDTNPIFLFNKEMILCDRAPAIPKATFSTENDMEIKVEESLMMPAVFHTVASRTQLAVEMYDVAKKLCSFCEGLVHDEHLQHQGWAAIMANLEDCSNSYQKLLFKFESIYSDYLQSIEDIKLKLTHLGTAVSVMAKIPLLECLTRHSYRECLGRPDSLNEHEGSEKAEMKRSTELVLSPDMPRTTNTSLVTSFHKSMEHVAPDPTGTERGKELRESCQSTVQQEEASVDAKDSDLPFFNVSLLDWINVQDRPNDVESLVRKCFDSMSRLDPKIIQPFMLECHQTIAKLDNQNMKAIKGLEDRLYALDQMIASCSRLVNEQKELAQGFLANQMRAENLKDASVLPDLCLSHANQLMIMLQNHRKLLDIKQKCTTAKQELANNLHVRLKWCCFVMLHADQDGEKLQALLRLVIELLERVRIVEALSTVPQMYCLAVVEVVRRKMFIKHYREWAGALVKDGKQLYEAEKSKRESFGKLFRKSFLRNRLFKGLDSWPSSFCTQKPRKFDCELPDISLKDLQFLQSFCPSEVQPFLRVPLLCDFEPLHQHVLALHNLVKAAQSLDEMSQTITDLLNEQKVSTSQASPQSAASPRIESTTGITTTTSPKTPPPLTVQDTLCPAVCPLEELSPDSIDAHTFDFETISHPNTEQPVHQASIDLDSLAESPESDFMSAVNEFVIEENLSSPNPISDPQSPEMMVESLYSSVINAIDSRRMQDTSTRGNEGFGDRAALHVQLEKCRAAAQDSHTSIQTIKDDLCHFRTFVQKEQCDLANYLKCTAVEIRNIIEKVKCSLEITLKEKHQQELQSLKIEYECKLDALVKDSEENVNKILKLKENLVSLEEALQNKDNEFTSIKHEKDAIVCVQQEKDQKLLEMEKIMHTQHCEIKELKQSREMALEDLKKLHDEKIESLRAEFQCLEENHLKELEDTLHIRHTQEFEKVMTDHNMSLEKLKKENQQRIDQMLESHASTIQEKEQQLQELKLKVSDLSDMRCKLEVELALKEAETDEIKILLEESRTQQKEMLKSLLEQETENLRTEISKLNQKIHDNNESYQVGLSELRALMTIEKDQCISELISRHEEESNILKAELDNVTSLHRQAYEIEKKLKEQIVELQTRLNSELSALEKQKDEKITQQEEKYEALIQNLEKDKERLVKNHEQDKEHLIQELNFEKNKAVQTALDEFKVERELVEKELLEKVKHLENQIAKTPAFESAREDSSSLVAELQEKLQEEKAKFLEQLEEQEKRKNEEMQNVRTSLIAEQQTNFNTVLTREKMRKENIINDLSDKLKSTMQQQERDKDLIESLSEDRARLLEEKKQLEEEVSKLRTSSFLSSAPVAAAPELYGACAPELPGEPERSVMETADEGRLDSAMETSMMSVQENMLSEEKQRIMLLERTLQLKEEENKRLNQRLMSQSLSSVSSRHSEKIAIRDFQVGDLVLIILDERHDNYVLFTVSPTLYFLHSESLPALDLKPGEGASGASRRPWVLGKVMEKEYCQAKKAQNRFKVPLGTKFYRVKAVSWNKKV

SEQ ID NO: 3 - DNA, 6636 bp, Unknown
  human RB1CC1 gene
gtcgacaataacaaaccaagccgcggcggtgtccgcggccctgccgagccctcggcgttgcctcagaatcccccagtcgcctgggcccctcggctctgacaggccgcggccttctgtcccccggccccagacccagagccgaggggcctgctcgcgtccttgtccgcccggacccctccctgcctcctagagttcggggccgcggcgggcgggcgcccgggacgccggcggttgtgtcggcttagcggtgccgaatgggcggttggtaaccgctgccgaggactaggcggcggcggaagatggtgccgggggtcgctggctctgctgctgccgccggcgaaggaggaggcgttgccggttttctgagtttaaccagtaatgccattcagttgccaatctcaagcaaagcaaacataagccagttttaatctactttttaagaaaagtggtagtccttttcacagtgcctgacgtaactgtatcagagggtgaggtataagctcacagaattcagataaatcatcatgaagttatatgtatttctggttaacactggaactactctaacatttgacactgaacttacagtgcaaactgtggcagaccttaagcatgccattcaaagcaaatacaagattgctattcaacaccaggtgctggtggtcaatggaggagaatgcatggctgcagatcgaagagtgtgtacctacagtgctgggacggatacaaatccaatttttctttttaacaaagaaatgatcttatgcgatcgtccacctgctattcctaaaactaccttttcgacagaaaatgacatggaaataaaagttgaagaatctcttatgatgcctgcagtttttcatactgttgcttcaaggacacagcttgcattggaaatgtatgaagttgccaagaaactttgttctttttgtgaaggtcttgtacatgatgaacatcttcaacaccaaggctgggctgcaatcatggccaacctggaggactgttcaaattcataccaaaagctacttttcaagtttgaaagtatttattcaaattatctgcagtccatagaagacatcaagttaaaacttactcatttaggaactgcagtttcagtaatggccaagattccactgttggagtgcctaaccagacatagttacagagaatgtttgggaagactggattctttacctgaacatgaagactcagaaaaagctgagacgaaaagatccactgaactggtgctctctcctgatatgcctagaacaactaacgaatctttgttaacctcatttcccaagtcagtggaacatgtgtccccagataccgcagatgctgaaagtggcaaagaaattagggaatcttgtcaaagtactgttcatcagcaagatgaaactacgattgacactaaagatggtgatctgcccttttttaatgtctctttgttagactggataaatgttcaagatagacctaatgatgtggaatctttggtcaggaagtgctttgattctatgagcaggcttgatccaaggattattcgaccatttatagcagaatgccgtcaaactattgccaaacttgataatcagaatatgaaagccattaaaggacttgaagatcggctctacgccctggaccagatgattgctagctgtggccgactggtgaatgaacagaaagagcttgctcagggatttttagctaatcagaagagagctgaaaacttaaaggatgcatctgtattacctgatttatgcctgagtcacgcaaatcagttgatgattatgttgcaaaatcatagaaaactgttagatattaagcagaagtgtaccactgccaaacaagaactagcaaataacctacatgtcagactgaagtggtgttgctttgtaatgcttcatgctgatcaagatggagagaagttacaagctttgctccgcctcgtaatagagctgttagaaagagtcaaaattgttgaagctcttagtacagttcctcagatgtactgcttagctgttgttgaggttgtaagaagaaaaatgttcataaaacactacagggagtgggctggtgctttagtcaaagatggaaagagattatatgaagcagaaaaatcaaaaagggaatcctttgggaaattatttaggaagtcttttttaagaaatcgtctgtttaggggactggactcctggcccccttccttttgtactcaaaagcctcgaaagtttgactgtgaacttccagatatttcattaaaagatttacagtttctgcaatcattttgtccttcggaagttcagccattcctcagggttcccttactttgtgactttgaacctctacaccagcatgtacttgctctacataatttggtaaaagcagcacaaagtttggatgaaatgtcacagaccattacagatctactgagtgaacaaaaggcatctgtgagccagacatccccacagtctgcttcttcaccaaggatggaaagtacagcaggaattacaactactacctcaccgagaactcctccaccactgactgttcaggatcccttatgtcctgcagtttgtcccttagaagaattatctccagatagtattgatgcacatacgtttgattttgaaactattccccatccaaacatagaacagactattcaccaagtttctttagacttggattcattagcagaaagtcctgaatcagattttatgtctgctgtgaatgagtttgtaatagaagaaaatttgtcgtctcctaatcctataagtgatccacaaagcccagaaatgatggtggaatcactttattcatcagttatcaatgcgatagacagtagacgaatgcaggatacaaatgtatgtggtaaggaggattttggagatcatacttctctgaatgtccagttggaaagatgtagagttgttgcccaagactctcacttcagtatacaaaccattaaggaagacctttgccactttagaacatttgtacaaaaagaacagtgtgacttctcaaattcattaaaatgtacagcagtagaaataagaaacattattgaaaaagtaaaatgttctctggaaataacactaaaagaaaaacatcaaaaagaactactgtctttaaaaaatgaatatgaaggtaaacttgacggactaataaaggaaactgaagagaatgaaaacaaaattaaaaaattgaagggagagttagtatgccttgaggaggttttacaaaataaagataatgaatttgctttggttaaacatgaaaaagaagctgtaatctgcctgcagaatgaaaaggatcagaagttgttagagatggaaaatataatgcactctcaaaattgtgaaattaaagaactgaagcagtcacgagaaatagtgttagaagacttaaaaaagctccatgttgaaaatgatgagaagttacagttattgagggcagaacttcagtccttggagcaaagtcatctaaaggaattagaggacacacttcaggttaggcacatacaagagtttgagaaggttatgacagaccacagagtttctttggaggaattaaaaaaggaaaatcaacaaataattaatcaaatacaagaatctcatgctgaaattatccaggaaaaagaaaaacagttacaggaattaaaactcaaggtttctgatttgtcagacacgagatgcaagttagaggttgaacttgcgttgaaggaagcagaaactgatgaaataaaaattttgctggaagaaagcagagcccagcagaaggagaccttgaaatctcttcttgaacaagagacagaaaatttgagaacagaaattagtaaactcaaccaaaagattcaggataataatgaaaattatcaggtgggcttagcagagctaagaactttaatgacaattgaaaaagatcagcgtatttccgagttaattagtagacatgaagaagaatctaatatacttaaagctgaattaaacaaagtaacatctttgcataaccaagcatttgaaatagaaaaaaacctaaaagaacaaataattgaactgcagagtaaattggattcagaattgagtgctcttgaaagacaaaaagatgaaaaaattacccaacaagaagagaaatacgaagctattatccagaaccttgagaaagacagacaaaaattggtcagcagccaggagcaagacagagaacagttaattcagaagcttaattgtgaaaaagatgaagctattcagactgccctaaaagaatttaaattggagagagaagttgttgagaaagagttattagaaaaagttaaacatcttgagaatcaaatagcaaaaagtcctgccattgactctaccagaggagattcttcaagcttagttgctgaacttcaagaaaagcttcaggaagaaaaagctaagtttctagaacaacttgaagagcaagaaaaaagaaagaatgaagaaatgcaaaatgttcgaacatctttgattgcggaacaacagaccaattttaacactgttttaacaagagagaaaatgagaaaagaaaacataataaatgatcttagtgataagttgaaaagtacaatgcagcaacaagaacgggataaagatttgatagagtcactttctgaagatcgagctcgtttgcttgaggaaaagaaaaagcttgaagaagaagtcagtaagttgcgcagtagcagttttgttccttcaccatatgtagctacagccccagaactttatggagcttgtgcacctgaactcccaggtgaatcagatagatccgctgtggaaacagcagatgaaggaagagtggattcagcaatggagacaagcatgatgtctgtacaagaaaatattcatatgttgtctgaagaaaaacagcggataatgctgttagaacgaacattgcaattgaaagaagaagaaaataaacggttaaatcaaagactgatgtctcagagcatgtcttcagtatcttcaaggcattctgaaaagatagctattagagattttcaggtgggagatttggtactcatcatcctagacgaacgccatgacaattatgtgttatttactgttagtcctactttatattttctacattcagagtctctacctgccctggatctcaaaccaggtgagggtgcttcaggtgcatctagaagaccctgggtacttggaaaagtaatggaaaaagaatactgtcaagccaaaaaggcacaaaacagatttaaagttcctttggggacaaagttttacagagtgaaagccgtatcatggaataagaaagtataacttatggacaaaattaatacattctatgacatttttttctgatttgtcctgcagtgctcattcatcactccaaaaacagcaggccatctttttatgcaaaagtcagcgtgacaatatacttcactggtgtacatcgtttactttttaactggcttcattttaggaataataaattcatcagaatccttggctgaattaaaatggtttttgttttttggtttttttttttacccagacaactctagaaatgcggaccaaactacttcattttctcaaagggcataccttgtgcattgtggcttatgatgagccatattaattgcctgttaaatatacactagcttgaacttagatgttaaatgttattattaccagcatttgtccttttgtgaaatcagtatcagaatacttgcactctttaacacattctttataaaatgtataaattattcagaactatttaaaataaagaggagtgttattgcatgctgataatcattttgagtttgcctcagtagatactaaagcaaattgtttcagtttttttaaatgccctttgatgtttcaaaaaaaaaaaggaactgtaatttgattgactgattttaagatcagccataagtaatcagcaatcttcaaaagcactttcagtggattggtcatctgggttctaaagggaagagtctgtgctactaaccatttcaaatgcagactcaaaccttcccaacatctttatgactctagaataatcatattgatgaaatcgtaattcatggttgagtttcagaacaaaagatattcattgcacattaaccatttagaggtcatttaaataacaaaatattgtattgtaaaagaactgtacaattttaaaacaataaagatttgaacctgtaaatgtgtgtgccttttaaagaaggatacatttttaatatatttgagtgattgctgggaagtgtgaaaatattgttatgtatcatatcaaagagaaacatgtttattacaaaaatgttctttaactatatactatgtaacagggtaaacagtgttatgtagaatagaattgtgtaaactagatctttagagaagttgccattgagcaaagttatttaaatgagttagttgagttggatgagaattgtttgaggtttgttgctagagaacaataataaaataattctttttcagaaaatatttaatttcttcataaaaataagttaaatatttttttaaatatgtatatctaatagtacaaaatggaataaacatcatagtgtatagaaaactgaatttgacaagttaatgaataaatgaacaaatgatttcaaaa

SEQ ID NO: 4 - DNA, 6518 bp, Unknown
  mouse Rb1cc1 gene
ccgagtcgacaataacaaaccccacggcggccgcgacccagccctgccaagctctcagtgcctcggccggcggactcgggtccccgcgcggagccgaggggccggagcagcggctgcgcccgactcccatccttccgggcctcgccgggtactcggcggctgggcgccgacggttgtgtcggttggcggcgccgcaggggcggttgatagccgccgccgaggccagggcggcggcagaagatggtgcggagggccgccggctgtgttgctgccgcgggcggaggaggcgctgccggttctctgagtttcaccagtaatgccactcagttgccaatatcaagcaagcgcatataagacaattgtaatcttttaagaaaagtagtacttctcttcacagtatctggcggatcaacactggagggtgaggtgtcagcttccagaaagatcatcatgaagttatatgtgtttctggttaacaccggaaccacgctgacatttgacactgagctaactgtgcaaactgtggctgatcttaagcatgccattcaaagcaaatacaagattgctattcagcaccaggttctggtggtcaatggaggagaatgcatggctgcagatcgaagagtgtgtacttacagcgctgggacggacacaaatccaatttttctttttaataaagaaatgatcttatgtgaccgtgcacctgctattcctaaagctaccttttcaacagaaaatgacatggaaataaaagttgaagagtctcttatgatgcctgcagttttccacactgttgcttcaaggacacagcttgcagtggaaatgtatgacgttgccaagaagctctgctctttctgtgaagggcttgtccatgatgaacatcttcagcaccaaggctgggctgcaatcatggccaatctggaggactgttcaaattcataccaaaaacttcttttcaagtttgaaagtatttattctgattatcttcaatccatagaagacatcaagttaaaacttactcatttaggaactgctgtttcagtaatggccaagattccactattggagtgcctaaccagacatagttacagggaatgtttgggaagaccggattctttgaatgaacatgaaggctcagagaaagctgagatgaaaagatctactgaactggtgctctctcctgatatgcctagaacaacgaacacatccttggtaacctcatttcacaagtcaatggagcatgtagctccagatcccaccggtactgaacgtggcaaagaacttagggaatcttgtcaaagtactgtccagcaagaagaagcttcagtggatgctaaagacagtgatctgcctttttttaatgtttctttgttagactggataaatgttcaagatagacccaatgatgtggaatctctggtcaggaagtgctttgattctatgagcaggcttgacccaaagattattcaaccatttatgttagaatgccatcaaactattgccaaacttgataatcagaatatgaaagccattaaagggcttgaagatcggctgtatgccttggaccagatgattgctagctgtagccggctggtaaatgaacagaaagagcttgctcagggatttttagctaatcagatgagagctgaaaacttgaaggatgcatctgtgttacctgatctgtgtctgagtcatgcaaatcaactaatgattatgttgcaaaaccacagaaaactgttggatattaaacagaagtgcaccactgccaaacaagagctagcaaacaatctccacgtcagactgaagtggtgttgttttgtgatgcttcatgctgatcaagatggagaaaaactgcaggcactgctccgccttgtaatagagctgttagaaagagtcagaattgttgaggctcttagtacagttcctcagatgtattgcctagctgttgttgaggttgtaagaagaaaaatgttcattaaacactacagagagtgggctggtgctttagtcaaagacggaaaacaactatatgaagctgaaaagtcaaaaagggaatcctttgggaaattatttaggaagtcctttttaagaaatcgtctgtttaaaggactggactcctggccttcctcattttgtactcagaagcctcgaaaatttgactgtgaacttccagatatatcattaaaagatttacagtttcttcaatcattttgtccttcagaagtgcagccattcctcagggtccccttactttgtgactttgaacctctacaccagcatgtacttgccctacataatttggtaaaagcagcacaaagtttggatgaaatgtcacagactattacagatctcctaaatgaacaaaaggtatccacaagtcaggcatccccacagtcagctgcttctccaagaatagaaagtacaacaggcattacaaccactacctcaccaaaaactcctcctccactaactgttcaggacaccttatgtccggcagtgtgtcccttagaagaattatctccagatagtatcgatgctcatacatttgatttcgaaaccatctcccatccaaacacagaacaacctgttcaccaagcttctatagacttggattcattagcagaaagccctgagtctgactttatgtctgctgtgaatgagtttgtgatagaagaaaatttatcgtctccaaaccctataagtgatccacaaagtccagaaatgatggtggagtcactttactcttcagtcatcaatgcaatagatagtaggcgtatgcaagacacaagtacacgtggaaacgagggctttggggatcgggctgctctacatgtccagctggagaaatgcagagctgctgcacaagactctcacaccagtatacaaaccatcaaggacgatctgtgccatttcagaacatttgtacaaaaagaacagtgtgacttagcaaattatttaaaatgtacagctgtagaaataagaaatattattgaaaaagtaaaatgttctctagaaataacactaaaggaaaagcatcagcaagaactccaatctttaaaaattgagtatgaatgtaaacttgatgctctagtaaaagacagtgaagaaaatgtaaataaaattttaaaattgaaagaaaatttagtatcccttgaagaggctttacaaaataaagacaatgaattcacttcgattaaacatgaaaaggatgctattgtctgtgtgcagcaagaaaaggatcagaagttgttagagatggaaaagataatgcatactcaacattgtgaaattaaagaactgaagcagtcacgagagatggcattagaagacctgaaaaagctgcatgatgaaaaaatcgagtcattgagagctgaatttcagtgcttagaagaaaatcacctgaaggaattagaggacacactgcacatcaggcacacacaggagtttgagaaagttatgacagaccacaatatgtctttggagaaattaaaaaaagaaaatcagcaaagaattgaccagatgctagaatctcatgcctcaactattcaggaaaaagagcaacagctgcaggagttgaaactcaaagtttctgacttgtcagacatgagatgtaagttagaggttgaacttgcactaaaggaagcagaaacagatgagataaagatcttgttggaagagagcagaacacagcagaaggaaatgctgaagtctttacttgaacaagagaccgaaaacttaagaacagaaataagtaaactaaaccaaaaaattcatgataataatgagagttaccaggtgggtttgtcagagttaagagctttaatgacaattgaaaaagatcagtgcatttcagagttaatcagtagacatgaagaagaatctaatatacttaaggctgaattagacaatgttacatctttgcatcgccaagcatatgaaatagaaaaaaaactgaaagaacaaatagttgaattgcagactagattgaactcagaattgagtgctcttgaaaaacagaaagatgaaaaaattacccaacaagaagagaagtatgaagcacttatccagaaccttgagaaagacaaggagagactggtcaagaaccacgagcaagacaaagaacacttaattcaggagcttaattttgaaaaaaacaaagctgttcaaactgcactagatgaatttaaggtggagagagaacttgttgagaaagagttattagaaaaagttaaacatcttgagaatcaaatagccaaaactcctgcctttgagtcagccagagaagattcttcaagcttagttgcggaacttcaagagaaacttcaagaagaaaaagctaagtttctggaacaacttgaagaacaagagaaaagaaagaatgaggaaatgcaaaatgtcagaacctctttgattgctgagcagcagaccaactttaacacagtcttaacaagagagaaaatgaggaaagaaaacataataaatgatcttagtgataagctaaaaagtacaatgcagcagcaagagcgggataaagatttgatagagtcgctctctgaggaccgagctcgtttgcttgaagagaagaagcagcttgaagaggaagtgagtaaactccgcactagcagttttctttcctcagcacctgtggctgcagccccagagctctatggtgcgtgtgcacctgagctcccaggggagccagagagatcagtcatggagacggcagatgaaggaagactggattccgcaatggagacaagcatgatgtctgtccaagaaaacatgttatctgaagagaagcagaggatcatgctcctagaacggacattgcagttgaaagaagaagaaaacaagcggttaaatcaaagactgatgtctcagagtttgtcctcagtctcttcaaggcattctgaaaaaatagccattagagattttcaggtgggagatttggttctcatcatcctagatgagcggcacgacaattatgtattgtttactgttagtcctactttatattttctgcactcagagtctcttcctgccctggatctcaaaccaggtgagggagcttcaggtgcatctagaagaccctgggtccttgggaaagtaatggaaaaggaatactgtcaagccaaaaaggcacaaaacagatttaaagttcctttggggacaaagttttacagagtgaaagctgtgtcatggaataagaaagtatagccacagaagaaatctctacatctcataccatttttgatttgtcctccagtgctgataaactactctaaaaacagctggccattgttgggtttttttttgttgtttgtttgtttgtttgtttttacaaaagtcaacataacaatatacttcattggtggactgcacttaccttttaagtggctacatcttaggaacaataaatttattaaaattcttggctgaatcaaaatggttttgttttgtttccacccaaataactagaaattcggaccaaaatagatgttttccaagggcagagcctgcactgtggcttgtgactagcctcattagttgcctgttaataaacattagctgaatagttaccagtgttgttaccagcatttgtcctcttgtgaattcaagagtcctcgcactctttaacatgttctttataaaatgtataaacccttccaaactatttaaagaggagtgttattgcatgcagataatcataattttgagtttgcctcagaagactactaaagcaaatttgttcatttttttttaaaaaaatgccctttaatgtttcaaaaaaaaataacagtgtaatttgactgactttaagatcagccataaataatgagcagtcttcaaaagcacttttcacacagatcatctgggctccagggaggaagagtctgtgccactgatgttttcaagtgcaggactcactcaaacctctcagcatcttaggactgtttcaagtaatcatattgatgaactcgtaattcatggttgaccttcagaagaagatattcattgtatattaacatttagaggtcatttaaataacaaaagtctgtattgtaaaggacctgtacaattttaagacaataaagaattgaaagtgtaaatgtgtgtgccttttaaaggttacattttaaatatattgcgtgatttctgggaaaggtgaaaaaaatgttctgtatcaaagagaaacctgtttattaaaaaatgttgtttgtatcctatgtaacagggtgaagtggtgttctgtggaacagaaccatgtaaactcaaggtttaaaagctggcactgaacaaagatattgaagtagctaggctagttgattggaaagagtttcttcagggttgttgttagcagtaataaatgattctttttcagaaatatttaatttctccataaaaataagttggatatttttataaatatgtaatctaatagaatgaaaatggaataaacatagtgtatagaatacctaattcaaaaacatattaatgaataaacgaacaaatgatta

SEQ ID NO: 5 - DNA, 20 bp, Artificial
  artificially synthesized primer sequence called CC1-S1
gacgtaactgtatcagaggg

SEQ ID NO: 6 - DNA, 20 bp, Artificial
  artificially synthesized primer sequence called CC1-S2
tcagagggtgaggtataagc

SEQ ID NO: 7 - DNA, 28 bp, Artificial
  artificially synthesized primer sequence called CC1-S3
atcatcatgaagttatatgtatttctgg

SEQ ID NO: 8 - DNA, 21 bp, Artificial
  artificially synthesized primer sequence called CC1-ASP2
ttggccattactgaaactgca

SEQ ID NO: 9 - DNA, 20 bp, Artificial
  artificially synthesized primer sequence called CC1-S4
tgtggaatctttggtcagga

SEQ ID NO: 10 - DNA, 21 bp, Artificial
  artificially synthesized primer sequence called CC1-ASP1
taatccttggatcaagcctgc

SEQ ID NO: 11 - DNA, 21 bp, Artificial
  artificially synthesized primer sequence called CC1-S5
tgtaccactgccaaacaagaa

SEQ ID NO: 12 - DNA, 20 bp, Artificial
  artificially synthesized primer sequence called CC1-S6
cttcggaagttcagccattc

SEQ ID NO: 13 - DNA, 20 bp, Artificial
  artificially synthesized primer sequence called CC1-S6R
tccatccttggtgaagaagc

SEQ ID NO: 14 - DNA, 21 bp, Artificial
  artificially synthesized primer sequence called CC1-S7
tccccatccaaacatagaaca

SEQ ID NO: 15 - DNA, 21 bp, Artificial
  artificially synthesized primer sequence called CC1-S8
aaggaagacctttgccacttt

SEQ ID NO: 16 - DNA, 22 bp, Artificial
  artificially synthesized primer sequence called CC1-S9
gaactgaagcagtcacgagaaa

SEQ ID NO: 17 - DNA, 20 bp, Artificial
  artificially synthesized primer sequence called CC1-S10
ttgtcagacacgagatgcaa

SEQ ID NO: 18 - DNA, 22 bp, Artificial
  artificially synthesized primer sequence called CC1-S11
aaattatcaggtgggcttagca

SEQ ID NO: 19 - DNA, 20 bp, Artificial
  artificially synthesized primer sequence called CC1-S
ccaggagcaagacagagaac

SEQ ID NO: 20 - DNA, 20 bp, Artificial
  artificially synthesized primer sequence called CC1-AS
cgagctcgatcttcagaaag

SEQ ID NO: 21 - DNA, 20 bp, Artificial
  artificially synthesized primer sequence called CC1-AS5
tgcttgtctccattgctgaa

SEQ ID NO: 22 - DNA, 20 bp, Artificial
  artificially synthesized primer sequence called CC1-AS4
ccaaatctcccacctgaaaa

SEQ ID NO: 23 - DNA, 20 bp, Artificial
  artificially synthesized primer sequence called HCC22INTS
ctagacgaacgccatgacaa

SEQ ID NO: 24 - DNA, 23 bp, Artificial
  artificially synthesized primer sequence called HCC22INTAS
tggcttgacagtattctttttcc

SEQ ID NO: 25 - DNA, 21 bp, Artificial
  artificially synthesized primer sequence called CC1-AS2
tgagcactgcaggacaaatca

SEQ ID NO: 26 - DNA, 21 bp, Artificial
  artificially synthesized primer sequence called CC1-AS1
tgatgaatgagcactgcagga

SEQ ID NO: 27 - DNA, 20 bp, Artificial
  artificially synthesized primer sequence called RB1CC-RS1
cctccctgcctcctagagtt

SEQ ID NO: 28 - DNA, 20 bp, Artificial
  artificially synthesized primer sequence called RB1CC-R4
tagtcctcggcagcggttac

SEQ ID NO: 29 - DNA, 20 bp, Artificial
  artificially synthesized primer sequence called RB1CC-R3
aaactcagaaaaccggcaac

SEQ ID NO: 30 - DNA, 21 bp, Artificial
  artificially synthesized primer sequence called RB1CC-R2
tgccacagtttgcactgtaag

SEQ ID NO: 31 - DNA, 20 bp, Artificial
  artificially synthesized primer sequence called RB1CC-R1
tttccaatgcaagctgtgtc

SEQ ID NO: 32 - DNA, 21 bp, Artificial
  artificially synthesized primer sequence called RB1CC-RS2
gagtgaaagccgtatcatgga

SEQ ID NO: 33 - DNA, 21 bp, Artificial
  artificially synthesized primer sequence called RB1CC-RS3
aatgcggaccaaactacttca

SEQ ID NO: 34 - DNA, 20 bp, Artificial
  artificially synthesized primer sequence called on RB1CC-RS4
tcagtggattggtcatctgg

SEQ ID NO: 35 - DNA, 20 bp, Artificial
  artificially synthesized primer sequence called RB1CC-RS5
tgattgctgggaagtgtgaa

SEQ ID NO: 36 - DNA, 23 bp, Artificial
  artificially synthesized primer sequence called RB1CC-RS6
gagaattgtttgaggtttgttgc

SEQ ID NO: 37 - DNA, 20 bp, Artificial
  artificially synthesized primer sequence called RB1CC-R5
ttgctcaatggcaacttctc

SEQ ID NO: 38 - DNA, 23 bp, Artificial
  artificially synthesized primer sequence called MMK3-1-S
gggtgaggtataagctcacagaa

SEQ ID NO: 39 - DNA, 21 bp, Artificial
  artificially synthesized primer sequence called MMK3-2-S
tcttatgtgatcgtccacctg

SEQ ID NO: 40 - DNA, 23 bp, Artificial
  artificially synthesized primer sequence called MMK3-2-AS
caaaggattccctttttgatttt

SEQ ID NO: 41 - DNA, 26 bp, Artificial
  artificially synthesized primer sequence called MMK6-2-S
gctaatcagaagagagctgaaaactt

SEQ ID NO: 42 - DNA, 20 bp, Artificial
  artificially synthesized primer sequence called MMK1-1-S
gatggtgatctgcccttttt

SEQ ID NO: 43 - DNA, 20 bp, Artificial
  artificially synthesized primer sequence called MMK1-2-S
taagcatgccattcaaagca

SEQ ID NO: 44 - DNA, 23 bp, Artificial
  artificially synthesized primer sequence called MMK15-1-S
tttctttatacagggaagtcttt

SEQ ID NO: 45 - DNA, 21 bp, Artificial
  artificially synthesized primer sequence called MMK15-2-AS
ttttcagaatgccttgaagat

SEQ ID NO: 46 - DNA, 23 bp, Artificial
  artificially synthesized primer sequence called MMK31-2-S
aggataccatcatcctagacgaa

SEQ ID NO: 47 - DNA, 20 bp, Artificial
  artificially synthesized primer sequence called MMK31-2-AS
cttaccaccctcacctggtt

SEQ ID NO: 48 - DNA, 26 bp, Artificial
  artificially synthesized primer sequence called MMK40-S
ttttgtattttaagtttaggaactgc

SEQ ID NO: 49 - DNA, 23 bp, Artificial
  artificially synthesized primer sequence called MMK38-S
ataggatacaaatccaatttttc

SEQ ID NO: 50 - DNA, 27 bp, Artificial
  artificially synthesized primer sequence called MMK31-S
aaaatataggatacaaatccaatgaca

SEQ ID NO: 51 - DNA, 24 bp, Artificial
  artificially synthesized primer sequence called MMK36-S
aaaggatgcatctgtattacctga

SEQ ID NO: 52 - DNA, 19 bp, Artificial
  artificially synthesized primer sequence called MMK340-S1
gccaacctggaggactgtt

SEQ ID NO: 53 - DNA, 23 bp, Artificial
  artificially synthesized primer sequence called MCC-S1
ggttctctgagtttcaccagtaa

SEQ ID NO: 54 - DNA, 20 bp, Artificial
  artificially synthesized primer sequence called MCC-S2
atcaacactggagggtgagg

SEQ ID NO: 55 - DNA, 28 bp, Artificial
  artificially synthesized primer sequence called EcoCC1S3
atcatcatgaagttatatgtgtttctgg

SEQ ID NO: 56 - DNA, 20 bp, Artificial
  artificially synthesized prmier sequnece called MCC-S7
gatgcctgcagttttccaca

SEQ ID NO: 57 - DNA, 20 bp, Artificial
  artificially synthesized primer sequence called MCC-S8
acgtggcaaagaacttaggg

SEQ ID NO: 58 - DNA, 20 bp, Artificial
  artificially synthesized primer sequence called MCC-AS4
gcttcttcttgctggacagt

SEQ ID NO: 59 - DNA, 20 bp, Artificial
  artificially synthesized primer sequence called MCC-S11
ccttggaccagatgattgct

SEQ ID NO: 60 - DNA, 21 bp, Artificial
  artificially synthesized primer seuqnece called MCC-S9
gggtccccttactttgtgact

SEQ ID NO: 61 - DNA, 20 bp, Artificial
  artificially synthesized primer sequence called MCC-AS8
catccaaactttgtgctgct

SEQ ID NO: 62 - DNA, 20 bp, Artificial
  artificially synthesized primer sequence called MCC-S12
tgctgcacaagactctcaca

SEQ ID NO: 63 - DNA, 21 bp, Artificial
  artificialy synthesized primer sequence called MCC-AS6
ggcacagatcgtccttgatgg

SEQ ID NO: 64 - DNA, 22 bp, Artificial
  artificially synthesized primer sequence called MCC-S13
tgaacttgcactaaaggaagca

SEQ ID NO: 65 - DNA, 20 bp, Artificial
  artificially synthesized primer sequence called MCC-AS7
cagcatttccttctgctgtg

SEQ ID NO: 66 - DNA, 24 bp, Artificial
  artificially synthesized primer sequence called MCC-S14
tgagtgctcttgaaaaacagaaag

SEQ ID NO: 67 - DNA, 21 bp, Artificial
  artificially synthesized primer sequence called MCC-S15
ttgcggaacttcaagagaaac

SEQ ID NO: 68 - DNA, 24 bp, Artificial
  artificially synthesized primer sequence called MIRB1CC-5
ctggaacaacttgaagaacaagag

SEQ ID NO: 69 - DNA, 20 bp, Artificial
  artificially synthesized primer sequence called MIRB1CC-3
acgagctcggtcctcagaga

SEQ ID NO: 70 - DNA, 20 bp, Artificial
  artificially synthesized primer sequence called MCC-S3
tcaggtgggagatttggttc

SEQ ID NO: 71 - DNA, 20 bp, Artificial
  artificially synthesized primer sequence called MCC-AS3
tgccgctcatctaggatgat

SEQ ID NO: 72 - DNA, 20 bp, Artificial
  artificially synthesized primer sequence called MCC-AS2
cagcactggaggacaaatca

SEQ ID NO: 73 - DNA, 20 bp, Artificial
  artificially synthesized primer sequence called MCC-AS1
agtcacaagccacagtgcag

SEQ ID NO: 74 - DNA, 20 bp, Artificial
  artificially synthesized primer sequence called MCC-ASR1
tgctttgaatggcatgctta

SEQ ID NO: 75 - DNA, 20 bp, Artificial
  artificially synthesized primer sequence called MCC-ASR2
cctcaccctccagtgttgat

SEQ ID NO: 76 - DNA, 18 bp, Artificial
  artificially synthesized primer seuqnece called MCC-ASR3
cctccgcaccatcttctg

SEQ ID NO: 77 - DNA, 18 bp, Artificial
  artificially synthesized primer sequence called INTRON1ASR
cagggtccccgtaggact

SEQ ID NO: 78 - DNA, 20 bp, Artificial
  artificially synthesized primer sequence called MCC-SR1
tcaggtgggagatttggttc

SEQ ID NO: 79 - DNA, 20 bp, Artificial
  artificially synthesized priemr sequence called MCC-SR2
ccagcatttgtcctcttgtg

SEQ ID NO: 80 - DNA, 21 bp, Artificial
  artificially synthesized primer sequence called MCC3=S3
ttttgagtttgcctcagaaga

SEQ ID NO: 81 - DNA, 20 bp, Artificial
  artificially synthesized primer sequence called MCC3-S4
tcggaattcatggttgacct

SEQ ID NO: 82 - DNA, 20 bp, Artificial
  artificially synthesized primer sequence called MCC3-AS2
tttcccagaaatcacgcaat

SEQ ID NO: 83 - DNA, 21 bp, Artificial
  artificially synthesized primer sequence called MCC3-AS13
tctttgttcagtgccagcttt

SEQ ID NO: 84 - DNA, 20 bp, Artificial
  artificially synthesized primer seuqnece called MCC15S
accaggtgggtttgtcagag

SEQ ID NO: 85 - DNA, 20 bp, Artificial
  artificially synthesized primer sequence called MCC15AS
cttggcgatgcaaagatgta

SEQ ID NO: 86 - DNA, 20 bp, Artificial
  artificially synthesized primer sequence called MCC-3S
acactggagggtgaggtgtc

SEQ ID NO: 87 - DNA, 20 bp, Artificial
  artificially synthesized primer sequence called MCC-3AS
gtgtcaaatgtcagcgtggt

SEQ ID NO: 88 - DNA, 18 bp, Artificial
  artificially synthesized primer sequence called MINT1-S
gacggttgtgtcggttgg

SEQ ID NO: 89 - DNA, 20 bp, Artificial
  artificially synthesized primer sequence called MINT1-AS
ttggcaactgagtggcatta

SEQ ID NO: 90 - DNA, 20 bp, Artificial
  artificially synthesized primer sequence called MINT2-S0
tgccactcagttgccaagta

SEQ ID NO: 91 - DNA, 22 bp, Artificial
  artificially synthesized primer sequence called MINT2-S
cacagtatctggcggtaagtca

SEQ ID NO: 92 - DNA, 20 bp, Artificial
  artificially synthesized primer sequence called MINT3-AS
cccagcgctgtaagtacaca

SEQ ID NO: 93 - DNA, 20 bp, Artificial
  artificially synthesized primer sequence called MINT4-S
taagcatgccattcaaagca

SEQ ID NO: 94 - DNA, 20 bp, Artificial
  artificially synthesized primer sequence called MINT4-AS
caggtgcacggtcacataag

SEQ ID NO: 95 - DNA, 20 bp, Artificial
  artificially synthesized primer seuqnece called MINT5-S
gcagttttccacactgttgc

SEQ ID NO: 96 - DNA, 20 bp, Artificial
  artificially synthesized primer sequence called MINT5-AS
ctccagattggccatgattg

SEQ ID NO: 97 - DNA, 20 bp, Artificial
  artificially synthesized primer sequence called MINT6-S
gccaatctggaggactgttc

SEQ ID NO: 98 - DNA, 20 bp, Artificial
  artificially synthesized primer seuqnece called MINT6-AS
agaatccggtcttcccaaac

SEQ ID NO: 99 - DNA, 21 bp, Artificial
  artificially synthesized primer sequence called MINT7-S
cccaatgatgtggaatctctg

SEQ ID NO: 100 - DNA, 20 bp, Artificial
  artificially synthesized primer sequence called MINT7-AS
agcaatcatctggtccaagg

SEQ ID NO: 101 - DNA, 19 bp, Artificial
  artificially synthesized primer sequence called MINT8-S
aagatcggctgtatgcctt

SEQ ID NO: 102 - DNA, 22 bp, Artificial
  artificially synthesized primer sequence called MINT8-AS
caacagttttctgtggttttgc

SEQ ID NO: 103 - DNA, 21 bp, Artificial
  artificially synthesized priemr sequence called MINT9-S
ggatgcatctgtgttacctga

SEQ ID NO: 104 - DNA, 20 bp, Artificial
  artificially synthesized primer sequence called MINT9-AS
gcctgcagtttttctccatc

SEQ ID NO: 105 - DNA, 20 bp, Artificial
  artificially synthesized primer sequence called MINT10-S
gctccgccttgtaatagagc

SEQ ID NO: 106 - DNA, 20 bp, Artificial
  artificially synthesized primer sequence called MINT10-AS
ccaaaggattccctttttga

SEQ ID NO: 107 - DNA, 20 bp, Artificial
  artificially synthesized primer sequence called MINT11-S
gggctggtgctttagtcaaa

SEQ ID NO: 108 - DNA, 20 bp, Artificial
  artificially synthesized primer sequence called MINT11-AS
aaaatgaggaaggccaggag

SEQ ID NO: 109 - DNA, 20 bp, Artificial
  artificially synthesized primer sequence called MINT12-S
actcctggccttcctcattt

SEQ ID NO: 110 - DNA, 20 bp, Artificial
  artificially synthesized primer sequence called MINT12-AS
tgaggaatggctgcacttct

SEQ ID NO: 111 - DNA, 21 bp, Artificial
  artificially synthesized primer sequence called MINT13-S
gcctcgaaaatttgactgtga

SEQ ID NO: 112 - DNA, 21 bp, Artificial
  artificially synthesized primer sequence called MINT13-AS
tccaaactttgtgctgctttt

SEQ ID NO: 113 - DNA, 21 bp, Artificial
  artficially synthesized primer sequence called MINT14-S
aaaagcagcacaaagtttgga

SEQ ID NO: 114 - DNA, 20 bp, Artificial
  artificially synthesized primer sequence called MINT14-AS
tggaggaggagtttttggtg

SEQ ID NO: 115 - DNA, 20 bp, Artificial
  artificially synthesized primer sequence called MINT15-S
ccacgagcaagacaaagaac

SEQ ID NO: 116 - DNA, 21 bp, Artificial
  artificially synthesized primer sequence called MINT15-AS
tccgcaactaagcttgaagaa

SEQ ID NO: 117 - DNA, 20 bp, Artificial
  artificially synthesized primer sequence called MINT16-S
tgaggaaatgcaaaatgtca

SEQ ID NO: 118 - DNA, 20 bp, Artificial
  artificially synthesized priemr sequence called MINT16-AS
gctcttgctgctgcattgta

SEQ ID NO: 119 - DNA, 22 bp, Artificial
  artificially synthesized primer sequence called MINT17-S
aaagtacaatgcagcagcaaga

SEQ ID NO: 120 - DNA, 21 bp, Artificial
  artificially synthesized primer sequence called MINT17-AS
tgctgaggaaagaaaactgct

SEQ ID NO: 121 - DNA, 19 bp, Artificial
  artificially synthesized primer sequence called MINT18-S
agctctatggtgcgtgtgc

SEQ ID NO: 122 - DNA, 21 bp, Artificial
  artificially synthesized primer sequence called MINT18-AS
gagcatgatcctctgcttctc

SEQ ID NO: 123 - DNA, 22 bp, Artificial
  artificially synthesized primer sequence called MINT19-S
tctgaagagaagcagaggatca

SEQ ID NO: 124 - DNA, 22 bp, Artificial
  artificially synthesized primer sequence calle MINT19-AS
cagtctttgatttaaccgcttg

SEQ ID NO: 125 - DNA, 24 bp, Artificial
  artificially synthesized priemr sequence called MINT20-S
cattgcagttgaaagaagaagaaa

SEQ ID NO: 126 - DNA, 21 bp, Artificial
  artificially synthesized primer sequence called MINT20-AS
tgccttgaagagactgaggac

SEQ ID NO: 127 - DNA, 22 bp, Artificial
  artificially synthesized primer sequence called MINT21-S
tctcttcaaggcattctgaaaa

SEQ ID NO: 128 - DNA, 20 bp, Artificial
  artificially synthesized primer sequence called MINT21-AS
atccagggcaggaagagact

SEQ ID NO: 129 - DNA, 20 bp, Artificial
  artificially synthesized primer sequence called MINT22-S
agcggcacgacaattatgta

SEQ ID NO: 130 - DNA, 22 bp, Artificial
  artificially synthesized primer seuqnece called MINT22-AS
ttttccattactttcccaagga

SEQ ID NO: 131 - DNA, 20 bp, Artificial
  artificially synthesized primer seuqnece called MINT23-S
ctgggtccttgggaaagtaa

SEQ ID NO: 132 - DNA, 20 bp, Artificial
  artificially synthesized primer seuqnece called MINT23-AS
cagcactggaggacaaatca

(19) United States
(12) Patent Application Publication
(10) Pub. No.: US 2006/0194293 A1
(43) Pub. Date: Aug. 31, 2006

(54) Rb1 gene induced protein (rb1cc1) and gene

(75) Inventors: Tokuhiro Chano, Shiga (JP); Hidetoshi Okabe, Kyoto (JP); Shiro Ikegawa, Tokyo (JP)

Correspondence Address: KILYK & BOWERSOX, P.L.L.C., 400 HOLIDAY COURT, SUITE 102, WARRENTON, VA, 20186, US

(73) Assignee: Tokuhiro CHANO, Shiga (JP)

(21) Appl. No.: 10/516,558

(22) PCT Filed: Jan. 30, 2003
(86) PCT No.: PCT/JP03/00882

§ 371 Date: Jan. 25, 2005

(51) Int. Cl. C12P 19/34

(52) U.S. Cl. 435/91.21

(57) ABSTRACT

To provide a novel gene and protein involved in multidrug resistance in cancer, to elucidate functions of the gene and protein, to provide methods of detecting the gene and antibody against the protein and of testing and diagnosing cancer using the gene and antibody, we found a novel protein (RB1CC1) or polypeptide and gene thereof present in nucleus of human or animal cells and having transcription factor functions and/or functions inducing expression of retinoblastoma-1 gene (RB1 gene) or the gene product. We determined the amino acid sequence and cDNA sequence, conducted gene amplification and detection with primers hybridizing with the gene, tested for expression and mutation of the gene, discovered the gene relates to cancer cell proliferation and assayed cancers, prepared antibody against the protein and detected the protein using the antibody, whereby we found a relation between the protein and cancer cell proliferation, and assayed cancers.

### TECHNICAL FIELD

The present invention relates to a novel protein and polypeptide (hereunder, referred to as “novel protein RB1CC1”) that can induce expression of a tumor-suppressor gene (retinoblastoma gene: RB1 gene). More specifically, the present invention relates to a polypeptide having all or a part of an amino acid sequence of a novel protein, a nucleic acid (hereunder, referred to as “RB1CC1 gene”) coding for the polypeptide, a recombinant vector containing the nucleic acid, a transformant that was transformed with the recombinant vector, a method for producing a peptide or polypeptide using the transformant, an antibody against the peptide or polypeptide, a method of screening for compounds that utilizes these, the screened compounds, an activity-inhibiting compound or activity-enhancing compound that acts on the polypeptide or the nucleic acid, a pharmaceutical composition relating to these, and a method of testing or diagnosing a disease relating to these as well as a reagent.

### BACKGROUND OF THE INVENTION

Multidrug resistance (MDR) that is resistance to treatment with anticancer agents is a major barrier to the successful treatments of cancer. While current understanding of factors that contribute to origins of MDR is limited, it is considered that P-glycoprotein that is a product of an MDR-associated gene (MDR1 gene) is involved in several cancers. It is also known that in other cancers expression of P-glycoprotein correlates inversely with emergence and metastasis of the cancer. It is considered that these different effects of P-glycoprotein are subject to suppression by different gene products or conduct different interactions. The identification of genes associated with MDR is essential in order to clarify these phenomena.

### SUMMARY OF THE INVENTION

A problem to be solved by the present invention is to discover a gene associated with multidrug resistance to anticancer agents as described above and the gene product thereof. More specifically, an object of the present invention is to provide a novel protein and polypeptide (novel protein RB1CC1) that can induce expression of the tumor-suppressor gene (retinoblastoma gene: RB1 gene). Another object of the present invention is to provide the nucleic acid (hereunder, “RB1CC1 gene”) coding for all or the part of the amino acid sequence of the novel protein, and the method for producing the protein or polypeptide (novel protein RB1CC1) using genetic engineering techniques. A further object of the present invention is to provide the antibody against the polypeptide derived from the novel protein RB1CC1. Other objects of the present invention are to conduct screening for an inhibitor, antagonist, or activator for actions of the novel protein RB1CC1 utilizing the aforementioned substances, to provide screened compounds, and to provide the pharmaceutical composition for use in treatment of multidrug resistance (MDR) that is resistance to treatment with anticancer agents utilizing these. Another problem to be solved by the present invention is to provide the method for diagnosing a cancer cells or cancer by testing for the novel protein and polypeptide (RB1CC1 protein) that can induce expression of the tumor-suppressor gene (retinoblastoma gene: RB1 gene) or the nucleic acid (hereunder, “RB1CC1 gene”) coding for all or a part of the amino acid sequence of the protein, that were clarified in the present invention. A further object of the present invention is to provide nucleic acid primers that can amplify a nucleic acid coding for all or the part of the amino acid sequence of the protein, and to provide the method for diagnosing cancer cells or cancer by testing for an amplification product of the nucleic acid using primers. A still further object of the present invention is to provide the antibody that can react with the protein or polypeptide (RB1CC1 protein), as well as an immunological assay method that uses the antibody. A further object of the present invention is to provide an assay reagent or kit that uses the primers or the antibody to be used in the assay method.

In order to solve the above problems, the present inventors identified a gene expressing differentially in U-2 OS osteosarcoma cells and MDR-variant induced cells and determined the nucleotide sequence thereof and the amino acid sequence encoded by cDNA of the novel protein. Further, in order to verify that a similar protein is present in animals, inventors determined the amino acid sequence of a novel protein in mouse and the amino acid sequence encoded by cDNA of the novel protein. In addition, inventors prepared antibodies that recognize these proteins and conducted immunological assay in addition to assay of expression, mutation, deletion and the like for the gene, and found that expression of the gene and expression of the protein are suppressed in certain kinds of cancer cells, thereby completing the present invention.

That is, the present invention comprises the following:
1. A protein or polypeptide which is present in the nucleus of human or animal cell and which has a function that can induce a transcription factor function and/or expression of retinoblastoma gene (RB1 gene) or a gene product thereof.
2. The human protein according to the above 1, which is a polypeptide or protein selected from the group consisting of: (1) a polypeptide or protein represented by an amino acid sequence described in SEQ ID No: 1 in the sequence listing; (2) a polypeptide containing an amino acid sequence comprising at least five amino acids of the amino acid sequence of the said polypeptide or protein; (3) a polypeptide or protein having homology of at least approximately 70% at the amino acid sequence level with the said polypeptide or protein; and (4) a protein or polypeptide having a mutation or induced mutation such as a deletion, substitution or addition of one to several amino acids relative to the amino acid sequence of the polypeptide or protein according to any one of the preceding (1) to (3).
3. The animal protein according to the above 1 that is a protein derived from mouse, which is a polypeptide or protein selected from the group consisting of: (1) a polypeptide or protein represented by an amino acid sequence described in SEQ ID No: 2 in the sequence listing; (2) a polypeptide comprising at least five amino acids of the amino acid sequence of the said polypeptide or protein; (3) a polypeptide or protein having homology of at least approximately 70% at the amino acid sequence level with the said polypeptide or protein; and (4) a protein or polypeptide having a mutation or induced mutation such as a deletion, substitution or addition of one to several amino acids relative to the amino acid sequence of the said polypeptide or protein according to any one of the preceding (1) to (3).
4. A nucleic acid coding for the polypeptide or protein according to any one of the above 1 to 3, or a complementary strand thereof.
5. A nucleic acid hybridizing under stringent conditions with the nucleic acid or the complementary strand thereof according to the above 3.
6. A nucleic acid represented by a base sequence comprising at least 15 consecutive bases of the base sequence of a nucleic acid described in SEQ ID No: 3 to 4 in the sequence listing or a complementary strand thereof, wherein a polypeptide expressed by transcription of the nucleic acid is the polypeptide according to any one of the above 1 to 3.
7. A recombinant vector containing the nucleic acid according to any one of the above 4 to 6.
8. A transformant that was transformed with the recombinant vector according to the above 7.
9. A method for producing the polypeptide or protein according to any of the above 1 to 3, comprising a step of culturing the transformant according to the above 8.
10. Nucleic acid primers represented by SEQ ID Nos: 5 to 132 in the sequence listing, which hybridize under stringent conditions with the nucleic acid or the complementary strand thereof according to any one of the above 4 to 6.
11. An antibody that immunologically recognizes the polypeptide or protein according to any one of the above 1 to 3.
12. A method of screening for compounds that inhibit or enhance a function that can induce transcription factor activity of the polypeptide or protein and/or expression of RB1 gene according to any of the above 1 to 3, wherein the method uses at least one member of the group consisting of the polypeptide or protein according to any one of the above 1 to 3 and the antibody according to the above 11.
13. A method of screening for compounds that interact with the nucleic acid according to the above 4 or 6 to inhibit or enhance expression of the nucleic acid, wherein the method uses at least one member of the group consisting of the nucleic acid according to anyone the above4 to 6, the vector according to the above 7, the transformant according to the above 8, and the nucleic acid primers according to the above 10.
14. A compound that was screened by the screening method according to the above 12 or 13.
15. A compound that inhibits or enhances a function that can induce transcription factor activity and/or expression of RB1 gene of the polypeptide or protein according to any of the above 1 to 3.
16. A compound that interacts with the nucleic acid according to any one of the above 4 to 6 to inhibit or enhance expression of the nucleic acid.
17. A pharmaceutical composition for use in treatment of multidrug resistance that is resistance to treatment with anticancer agents, wherein the pharmaceutical composition comprises at least one member of the group consisting of the polypeptide or protein according to any of the above 1 to 3, the nucleic acid according to any one of the above 4 to 6, the vector according to the above 7, the transformant according to the above 8, the nucleic acid primers according to the above 10, the antibody according to the above 11, and the compound according to any one of the above 14 to 16.
18. A method of testing or diagnosing a disease related with expression or activity of the polypeptide or protein according to any of the above 1 to 3, wherein the method comprises a step of conducting analysis employing (a) a nucleic acid encoding the polypeptide or protein and/or (b) the polypeptide or protein in a sample, as a marker.
19. The method of testing or diagnosing according to the above 18, which is a method of testing cancer cells or a method of diagnosing a cancer.
20. The method according to the above 18 or 19 which examines expression, increase, decrease, deletion or the like of all or a part of the polypeptide or protein according to any of the above 1 to 3, wherein the method uses the antibody according to the above 11.
21. The method according to the above 18 or 19 which examines expression, mutation, deletion or insertion or the like of all or a part of a gene encoding the polypeptide or protein according to any of the above 1 to 3 through a step of amplifying a gene encoding the polypeptide or protein according to any of the above 1 to 3 using at least one of nucleic acid primers according to the above 10.
22. The method according to any of the above 18 to 21, wherein the method combines examination of expression, increase, decrease, mutation, deletion or insertion or the like of all or a part of the tumor-suppressor gene retinoblastoma gene (RB1 gene) or the gene product thereof (RB1 protein).
23. The method according to any of the above 18 to 22, wherein the method combines examination of expression, increase, decrease, mutation, deletion or insertion or the like of all or a part of multidrug resistance gene (MDR1 gene) or the gene product thereof (MDR1 protein: P-glycoprotein).
24. The method according to any of the above 18 to 23, wherein the method combines examination of expression, increase, or decrease or the like of all or a part of the cell proliferation marker, Ki-67 protein.
25. A method that examines drug sensitivity of a cancer cell using the method according to the above 23.
26. A reagent and a kit for assay or diagnosis, for use in the method according to any of the above 18 to 25.

### BRIEF DESCRIPTION OF DRAWINGS

FIG. 1 shows photographs of Northern blots that examined the relation between expression of human RB1CC1 gene and MDR1 gene.

FIG. 2 shows a photograph of Western blots and of cellular immunostaining showing that human RB1CC1 protein is present in nucleus.

FIG. 3 shows photographs of Western blots and of cellular immunostaining showing that mouse Rb1cc1 protein is present in nucleus.

FIG. 4 is a diagram that examined the effect on cell proliferation resulting from treatment with the anticancer agent doxorubicin.

FIG. 5 shows photographs of Northern blots that examined the relation between cell proliferation caused by treatment with the anticancer agent doxorubicin and RB1CC1 gene expression and RB1 gene expression.

FIG. 6 is a photograph of electrophoresis of RT-PCR products that examined the relation between RB1CC1 gene expression and RB1 gene expression in various cancer cells.

FIG. 7 shows photographs of Northern blots that examined the relation between RB1CC1 gene expression and RB1 gene expression in various human organs.

FIG. 8 is a photograph of a Northern blot that examined the relation between RB1CC1 gene expression and RB1 gene expression in various mouse organs.

FIG. 9 is a photograph of electrophoresis of RT-PCR products that examined the effect on RB1 gene expression caused by introduction of RB1CC1 gene.

FIG. 10 is a diagram showing results obtained after testing the effect on transcriptional activity of RB1 gene promoter region caused by RB1CC1 gene induction.

FIG. 11 is a photograph of results obtained after testing loss of heterozygosity of RB1CC1 gene locus in a variety of primary breast cancers.

FIG. 12 shows a photograph of electrophoresis of RT-PCR products that examined mutation of RB1CC1 gene in primary breast cancers, and a view showing the results of gene sequence analysis.

FIG. 13 shows photographs of Western blots that examined expression of RB1CC1 protein and RB1 protein in primary breast cancers.

FIG. 14 shows photographs of immunohistological staining that examined expression of RB1CC1 protein and RB1 protein in primary breast cancers.

FIG. 15 shows diagrams illustrating the correlation between RB1CC1 as a stain indicator and Ki-67 and RB1.

### DETAILED DESCRIPTION OF THE INVENTION

(Novel Protein RB1CC1)

The cDNA of the nucleic acid encoding the novel protein RB1CC1 provided according to the present invention was obtained by identifying a gene expressing differentially in U-2 OS osteosarcoma cells and MDR-variant induced cells, conducting amplification employing U-2 OS mRNA as a template using nucleic acid primers described in SEQ ID Nos: 5 to 37 in the sequence listing, and determining the amino acid sequence coded for by cDNA of the novel protein and the base sequence, to thereby obtain the cDNA as a substance having a novel amino acid sequence. The cDNA of novel protein RB1CC1 of the present invention had a length of 6.6 kb, included an open reading frame (ORF) of 4782 nucleotides, and encoded a protein comprising 1594 amino acids with a molecular weight of 180 kDa.

The novel human protein RB1CC1 had a consensus nuclear localization signal sequence site (lysine-proline-arginine-lysine sequence: KPRK), a leucine zipper motif sequence site, and a coiled-coil structure. It was suggested that the novel human protein RB1CC1 has DNA-binding and transcription functions.

(Novel Mouse Protein Rb1cc1)

Amplification was conducted employing mRNA of mouse muscle as a template using the nucleic acid primers described in SEQ ID Nos: 53 to 83 in the sequence listing, and the amplification product was analyzed. The obtained cDNA coding for novel mouse protein Rb1cc1 had a chain length of 6518 bp with an open reading frame (ORF) of 4764 bp encoding 1588 amino acids. The novel mouse protein Rb1cc1 gene shared 89% homology with the novel human protein RB1CC1 gene. Similarly to the human protein, novel mouse protein Rb1cc1 had a consensus nuclear localization signal sequence site (lysine-proline-arginine-lysine sequence: KPRK), a leucine zipper motif sequence site, and a coiled-coil structure. It was suggested that mouse novel protein Rb1cc1 also has DNA-binding and transcription functions.

(Function of Novel Protein and Gene)

To investigate the role of RB1CC1 gene of the present invention in MDR, RB1CC1 gene expression was compared for cases in which doxorubicin treatment was conducted for parental U-2 OS cells, MDR variants of U-2 OS cells (U-2 OS/DX580), and U-2 OS cells introduced with MDR1 gene (U-2/DOXO35), whereby it was found that in the parental U2 OS cells and control cells introduced with a gene (U-2/Neo8) doxorubicin lowered expression of the RB1CC1 gene and induced cell death. In contrast, in the MDR variants of U-2 OS cells, doxorubicin treatment did not exhibit an inhibitory effect on the expression level of RB1CC1 gene, cell lifetime, or cell proliferation, and in cells with the MDR1 gene the RB1CC1 gene expression was increased. In these cells, RB1CC1 gene expression and RB1 gene expression correlated, and expression of both genes sustained the proliferation of these cells.

To examine the relation between expression of RB1 gene and the RB1CC1 gene of the present invention, expression of both genes in 5 kinds of MDR-variants of U-2 OS human osteosarcoma cells and 24 kinds of human tumor cells (10 kinds of osteosarcoma, 4 kinds of lung cancer, 7 kinds of breast cancer, 3 kinds of blood cancer) was examined, whereby it was found that RB1CC1 gene expression strongly correlated with RB1 gene expression in all of the cells. Expression of RB1CC1 gene and RB1 gene also showed a similar correlation in Northern blot analysis of nonneoplastic tissue.

Further, exogenous expression of the RB1CC1 gene of the present invention increased RB1 gene expression in K562 cells and Jurkat cells. Expression of MDR1 gene could not be detected in these cells. Induction of RB1CC1 gene also stimulated transcriptional activity of RB1 gene promoter. Introduction of the RB1CC1 gene raised expression of RB1 gene through the stimulated activity of the RB1 gene promoter.

Considering the amino acid sequence of the novel protein RB1CC1, the nuclear locality thereof, and the expression pattern thereof, there is a possibility that the RB1CC1 gene of the present invention is a transcription factor that enhances RB1 gene expression directly or indirectly through a molecular intermediate. While analysis of promoter sequences of RB1 genes derived from human and mouse indicates the possibility of the presence of a constitutive transcription factor such as Sp1 or ATF, a transcription factor that directly regulates RB1 gene expression is not known. In about 80% of human cancers, molecules that are present in the RB1 gene pathway are associated with the mechanism of carcinogenesis, and dysregulation of the RB1 gene plays an important role in the cancer of many people.

As shown in Table 1, human and mouse RB1CC1 genes of the present invention both contain 24 exons and 23 introns, and length 4 kb or more and 57 kb or more in human and mouse, respectively. A translation initiation position is present at the site of exon 3. The structure of the gene in mouse was clarified using primers set forth in SEQ ID Nos: 84 to 132 of the sequence listing. When we investigated the localization sites of the gene on a chromosome, we found that the gene is present at 8q11.2 on the chromosome 8 in human and at 1A2-4 on the chromosome 1 in mouse.

TABLE 1
Structure of RB1CC1 gene

| | |
|---|---|
| Exon | Intron |

| | | |
|---|---|---|
| nucleic acid | nucleic acid | |
| strand length (bp) | strand length (kb) | Human Sequence |

| | | | | | | | |
|---|---|---|---|---|---|---|---|
| No. | human | mouse | No. | human | mouse | receptor sequence in splicing | donor sequence in splicing |

| | | | | | | | |
|---|---|---|---|---|---|---|---|
| 1 | 358 | 298 | 1 | 9.1 | 11.2 | | GCGTTGCCGG gtaagtgtcg |
| 2 | 115 | 110 | 2 | 1.3 | 1.8 | tcttttccag TTTTCTCAGT | GTGCCTGACG gtaagtcaca |
| 3 | 122 | 115 | 3 | 1.4 | 3.5 | tttcttctag TAACTGTATC | CAGTGCAAAC gtaagttgta |
| 4 | 127 | 127 | 4 | 0.2 | 0.1 | ttttttgaag TGTGGCAGAC | TGCTGGGACG gtaggtattc |
| 5 | 171 | 171 | 5 | 7.0 | 3.8 | aaaaatatas GATACAAATC | GCTTGCATTG gtaagatata |
| 8 | 203 | 203 | 8 | 2.1 | 1.3 | ttcaatatag GAAATGTATG | AACTTACTCA gtatgtttgc |
| 7 | 430 | 427 | 7 | 5.7 | 3.8 | gtattttaag TTTAGGAACT | TATGAGCAGG gtaagtaacg |
| 8 | 171 | 171 | 8 | 6.3 | 0.5 | tgtcatttag CTTGATCCAA | GCTTGCTCAG gtacctattt |
| 9 | 185 | 185 | 9 | 0.3 | 0.2 | tttctcaaag GGATTTTTAG | TCAGACTGAA gtaagtgatt |
| 10 | 187 | 187 | 10 | 0.1 | 0.1 | tattctctag GTGGTGTTGC | CTACAGGGAG gtatgcaagt |
| 11 | 82 | 82 | 11 | 0.3 | 0.1 | cctcttctag TGGGCTGGTG | AAATTATTTA gtaagtgttc |
| 12 | 62 | 62 | 12 | 1.6 | 1.8 | ctttatacag GGAAGTCTTT | TTCCTTTTGT gtatgtattt |
| 13 | 104 | 104 | 13 | 0.8 | 0.3 | tttggtacag ACTCAAAAGC | CATTCCTCAG gtaaatgtca |
| 14 | 127 | 127 | 14 | 0.1 | 0.1 | tctgtttcag GGTTCCCTTA | TGAACAAAAG gcaaattcaa |
| 15 | 1801 | 1892 | 15 | 10.1 | 10.0 | tgttttccag GCATCTGTGA | TAGCAAAAAG gtaagaatta |
| 16 | 166 | 166 | 16 | 2.9 | 1.6 | aatttgtaag TCCTGCCATT | GGAACAACAG gtctgtatct |
| 17 | 109 | 109 | 17 | 0.1 | 0.1 | cttsttccag ACCATTTTA | CGGGATAAAG gtttgtactg |
| 18 | 241 | 241 | 18 | 6.3 | 1.1 | tgtccttcag ATTTGATAGA | TGTCTGTACA gtaagtatgg |
| 19 | 55 | 49 | 19 | 1.0 | 1.0 | tcacttttag AGAAAATATT | GTTAGAACGA gtaagtaaat |
| 20 | 48 | 48 | 20 | 4.4 | 3.0 | ccacctgcag ACATTGCAAT | TCAAAGACTG gtaagatttt |
| 21 | 59 | 59 | 21 | 2.3 | 2.1 | ttttttttag ATGTCTCAGA | CTATTAGAGA gtaagtattt |
| 22 | 137 | 137 | 22 | 3.5 | 2.0 | ctttattcag TTTTCAGGTG | GGTGAGGGTG gtaagtgtca |
| 23 | 71 | 71 | 23 | 0.8 | 1.6 | atttcattag CTTCAGGTGC | AGCCAAAAAG gtaaaaacga |
| 24 | 1401 | 1379 | | | | tccctcttag GCACAAAACA | |

Exon sequences are shown in upper case letters, and intron sequences are shown in lower case.

In order to detect mutations of RB1CC1 gene of the present invention, the RB1CC1 gene was analyzed using cDNA prepared from 35 cases of primary breast cancer, whereby 9 kinds of mutation were verified in 7 of cancers. There were lacks at exons 3 to 24 in all of 9 kinds of mutation, and the fragmented novel protein RB1CC1 had lost its consensus nuclear localization signal sequence site, leucine zipper motif sequence site and coiled-coil structure, and did not have functions of the fundamental novel protein RB1CC1.

Two of primary breast cancers (MMK 3 and 6) showed compound heterozygous lacks in both alleles, and it is predicted that a clearly fragmented novel protein RB1CC1 can be obtained from RB1CC1 gene with a lack. In MMK 6, there were lacks at exons 3 to 24 (nucleotides 534-5322) and exons 9 to 23 (nucleotides 1757-5187), with the respective frameshifting at codons 4 and 411. In MMK 3, there were lacks at exons 3 to 24 (nucleotides 535-5324) and exons 5 to 11 (nucleotides 849-2109), with termination occurring at codon 4 in the former, and a frame shift caused at codon 109 in the latter to result in obtainment of a protein fragment comprising 122 amino acids. Although irregular products corresponding to respective lack mutations were detected in PCR of genome DNA of cancer samples, mutations were not observed in DNA of embryonic cells, revealing that these mutations occur in somatic cells. The novel protein RB1CC1 was not detected in these cancers, and RB1 protein was absent in MMK 6 and was significantly less abundant than normal in MMK 3. There was no loss of heterozygosity at the RB1 loci on the chromosome in either case. In the cancer samples (MMK 12 and 29) without mutation of the RB1CC1 gene, both the novel protein RB1CC1 and RB1 protein were present. This suggests that inactivated mutation of the RB1CC1 gene causes RB1 gene expression to be insufficient and promotes dysregulation of the RB1 gene pathway, to cause canceration.

In other five breast cancers, (MMK 1, 15, 31, 38 and 40) also, lacks were detected in RB1CC1 gene that generated a protein fragment without function. These mutations were all heterozygotes, with loss of heterozygosity also present at the RB1CC1 loci, and since there was no expression of RB1CC1 gene in each of the cases, it was suggested that loss of function had occurred in both alleles. Expression of RB1 protein in these cancers was clearly reduced in comparison to cases (MMK 12 and 29) without mutation of RB1CC1 gene and RB1 gene. Loss of heterozygosity at the RB1 loci was not observed in these 5 cancers (MMK 1, 15, 31, 38, and 40).

Homozygous inactivation of the RB1CC1 gene of the present invention is associated with genesis of breast cancer. Lack mutations of the RB1CC1 gene that generated fragments of the novel protein RB1CC1 that clearly had no function were observed in approximately 20% of primary breast cancers examined. Two of these cancers showed plural heterozygous lacks within the RB1CC1 gene, and the remainder showed loss of heterozygosity of the RB1CC1 gene. Although the novel protein RB1CC1 could not be detected in any of seven cancers, protein was expressed in cancers without mutation of the RB1CC1 gene. Irrespective of the fact that there was no loss of heterozygosity at the RB1 loci, in all seven cancers the RB1 protein was either absent or significantly decreased.

The novel protein RB1CC1 performs regulation to increase expression of the RB1 gene, and the RB1CC1 gene functions as a tumor suppressor in breast cancer. Further, abnormality or inactivation of the RB1CC1 gene leads to a decline in expression of RB1 gene, causing genesis and progression of cancer.

As described in the above-mentioned, since expression of the RB1CC1 gene and protein correlate with expression of RB1 gene, a more useful method of diagnosing cancer cells or cancer can be provided by performing tests that combine testing for the RB1CC1 gene and protein of the present invention with testing for expression of the RB1 gene or expression of the protein.

Further, by also combining tests for multidrug resistance gene (MDR1) or the protein thereof, the effect of a pharmaceutical against a cancer or cancer cells can be examined, enabling the provision of an examination method or a diagnostic method that is useful for selecting an anticancer agent and predicting the effects thereof.

(Polypeptide or Protein)

The novel protein of the present invention is a polypeptide or protein comprising an amino acid sequence represented by SEQ ID No: 1 or 2 in the sequence listing. The polypeptide or protein of the present invention may also be selected from polypeptides having a partial sequence of the polypeptide represented by SEQ ID No: 1 or 2 in the sequence listing. The selected polypeptide preferably has homology of about 70% or more, more preferably about 80% or more, andfurther preferablyhas homologyexceeding about 90% with thepolypeptide representedby SEQID No: 1 or 2 in thesequence listing. Selection of polypeptides having the homology can be conducted, for example, by taking expression of RB1 gene or RB1 protein as an indicator.

Techniques for determining homology of an amino acid sequence are publicly known in the art and, for example, a method that directly determines the amino acid sequence or a method that first determines a putative base sequence of a nucleic acid and then predicts the amino acid sequence encoded by the base sequence may be used.

For the polypeptide of the present invention, an amino acid sequence selected from polypeptides having a partial sequence of a polypeptide or protein comprising an amino acid sequence set forth in SEQ ID No: 1 or 2 in the sequence listing can be utilized as a reagent, reference material or immunogen. The subject of the present invention is a polypeptide comprising, as a minimum unit thereof, the amino acid sequence composed of at least 5 amino acids, preferably at least 8 to 10 amino acids or more, and more preferably at least 11 to 15 or more amino acids which can be screened immunologically.

Further, by employing expression of RB1 gene or RB1 protein as the indicator, there can also be provided a polypeptide comprising an amino acid sequence having a mutation or induced mutation such as a deletion, substitution, addition or the like of one to several amino acids relative to the amino acid sequence of a polypeptide specified as described above. Methods for carrying out a deletion, substitution, addition or insertion are publicly known, and, for example, the technique of Ulmer (Science, 219: 666, 1983) can be utilized. These available peptides can also be modified to a degree that is not accompanied by a noticeable change in function, such as modification of constitutive amino groups or carboxyl groups or the like.

Polypeptides of the present invention can be used as they are in a pharmaceutical composition for regulating a function of the novel protein RB1CC1. Further, the polypeptide or protein of the present invention can be used in screening to obtain a compound that can regulate a function of the novel protein RB1CC1, for example, an inhibitor, antagonist, activator or the like, or an antibody against the novel protein RB1CC1. In addition, a polypeptide or protein of the present invention can also be used as a reagent or reference standard.

(Nucleic Acid)

The term “nucleic acid and a complementary strand thereof” of the present invention refers to a nucleic acid set forth in SEQ ID No: 3 or 4 in the sequence listing that codes for an amino acid sequence set forth in SEQ ID No: 1 or 2 in the sequence listing and the complementary strand for the nucleic acid, a nucleic acid hybridizing under stringent conditions with these nucleic acids, and a nucleic acid having a sequence of at least 15 consecutive base sequence derived from these nucleic acids in which a peptide encoded thereby is capable of binding with an antibody against the novel protein RB1CC1. When DNA is taken as a typical example of the nucleic acid, the term “DNA hybridizing under stringent conditions to DNA” refers to DNA that can be obtained by a publicly known method, for example, a method described in Molecular Cloning: A Laboratory Manual (Cold Spring Harbor Laboratory Press, 1989). Here the term “hybridizing under stringent conditions” refers to, for example, conditions under which a positive hybridization signal is still observed even after heating at 42° C. in a solution of 6×SSC, 0.5% SDS and 50% formamide, and washing at 68° C. in a solution of 0.1×SSC and 0.5% SDS.

The term “nucleic acid of the present invention” refers to a homologous strand and complementary strand selected from information of the nucleic acid set forth in SEQ ID No: 3 or 4 in the sequence listing that encodes an amino acid sequence described in SEQ ID No: 1 or 2 in the sequence listing, and also refers to a nucleic acid sequence comprising a sequence of at least about 15 to 20 nucleotides that correspond to a region of the specified nucleotide sequence, as well as the complementary strand thereof. Determination of this useful nucleic acid sequence can be conducted by simply confirming the expressed protein utilizing a publicly known protein expression system, for example, a cell-free protein expression system, and then screening by employing binding thereof with the antibody against bioactive novel protein RB1CC1 as the indicator. As the cell-free protein expression system, for example, a ribosome system derived from germ or rabbit reticulocyte or the like can be utilized (Nature, 179, 160-161, 1957).

Each of these nucleic acids provide genetic information that is useful for producing the novel protein RB1CC1 of the present invention and the polypeptide or protein of the present invention, and they can be used as primers or probes for detecting mRNA or a nucleic acid such as a gene encoding these, or as antisense oligomers to regulate gene expression. Further, a nucleic acid of the present invention can also be utilized as a reagent or reference standard relating to the nucleic acid.

(Transformant)

In addition to the cell-free protein expression system described above, by employing genetic recombination techniques using a publicly known host such as Escherichia coli, yeast, Bacillus subtilis, an insect cell or animal cell, it is possible to provide the novel protein RB1CC1 comprising the present invention and the polypeptide comprising a product derived therefrom.

Transformation can be conducted by applying publicly known means, for example, by transforming the host utilizing a plasmid, chromosome, virus or the like as a replicon. As a more preferable system, a method that conducts integration into the chromosome may be mentioned when considering genetic stability. However, as a simple and convenient method, an autonomous replication system using an extra nuclear gene can be utilized. A vector can be selected according to the kind of host, and gene sequences that are objects of expression and gene sequences carrying information relating to replication and regulation can be employed as constituent elements. Constituent elements can be selected according to whether the host is a prokaryotic cell or eukaryotic cell, and a promoter, ribosome binding site, terminator, signal sequence, enhancer and the like can be combined according to a publicly known method and used.

The transformant can be used to produce the polypeptide of the present invention by culturing the transformant after selecting optimal conditions from publicly known culture conditions for the respective hosts. While culturing may be conducted by employing as an indicator the physiological activity of the novel protein RB1CC1 to be expressed and produced and a polypeptide comprising the product derived therefrom, in particular, RB1 gene inducing activity or DNA-binding transcription factor activity, it is generally conducted by subculture or batch culture employing the quantity of transformant in the medium as an indicator.

(Recovery of the Novel Protein RB1CC1 and Product Derived Therefrom)

Recovery from the culture medium of the novel protein RB1CC1 and the polypeptide comprising the product derived therefrom can be conducted by carrying out purification and recovery that combines techniques such as a molecular sieving, an ion column chromatography, an affinity chromatography employing binding with the antibody against the novel protein RB1CC1 as the indicator, or by a fractionation technique using alcohol or ammonium sulfate or the like that is based on difference in solubility.

(Antibody)

An antibody can be prepared by screening for an antigenic determinant of the novel protein RB1CC1 of the present invention and the polypeptide comprising the product derived therefrom. The antigenic determinant is composed of at least five amino acids, and more preferably at least 8 to 10 amino acids. The amino acid sequence need not necessarily be homologous with SEQ ID No: 1 or 2 in the sequence listing, and it is sufficient that the sequence is a site that is exposed to outside of the tertiary structure of the protein. If the exposed site is a discontinuous site, it is also effective that the amino acid sequence that is continuous with respect to the exposed site. The antibody is not particularly limited as long as it immunologically recognizes the novel protein RB1CC1 and the polypeptide comprising the product derived therefrom. The presence or absence of the recognition can be determined by a publicly known antigen-antibody binding reaction.

Production of the antibody can be conducted by inducing immunity such as humoral response and/or cellular response in an animal using the novel protein RB1CC1 of the present invention and the polypeptide comprising the product derived therefrom by itself or in a state in which it is bonded with a carrier, in the presence or absence of an adjuvant. The carrier is not particularly limited as long as the carrier itself does not produce a deleterious effect on a host, and examples thereof include cellulose, polymerized amino acid, and albumin. As an animal to be immunized,mouse, rat, rabbit, goat, horse or the like is preferable. A polyclonal antibody can be obtained by a publicly known method for recovering antibody from serum.

Production of a monoclonal antibody can be carried out by recovering antibody-producing cells from the animal that has undergone the aforementioned immunization and introducing transformation means to publicly known constantly proliferating cells.

The polyclonal or monoclonal antibody can be bonded directly with the novel protein RB1CC1 of the present invention to enable control of the activity thereof, and control of expression of the novel protein RB1CC1 and RB1 gene or protein can be easily performed. Therefore, the antibody is useful for treating or preventing a disease with which the RB1 gene product and the novel protein RB1CC1 are associated.

(Screening)

According to the novel protein RB1CC1 and the polypeptide comprising the product derived therefrom that were prepared as described above, the nucleic acid encoding these and a complementary strand thereof, the cell transformed based on information of these amino acid sequences and base sequences, and the antibody that immunologically recognizes the novel protein RB1CC1 and the polypeptide comprising the product derived therefrom, by use of a single means or by combining a plurality of means, there can be provided means effective in screening for binding with the novel protein RB1CC1 and the polypeptide comprising the product derived therefrom, a function of the novel protein RB1CC1, or an inhibitor or activator of expression of the novel protein RB1CC1. More specifically, there can be provided a method of screening for compounds that inhibit or enhance expression of the polypeptide or protein and the RB1 gene or protein of the present invention by using at least one member of the group consisting of the polypeptide of the present invention and the antibody of the present invention. There can be provided a method of screening for compounds that interact with the nucleic acid of the present invention to inhibit or enhance expression of the nucleic acid by using at least one member of the group consisting of the nucleic acid of the present invention, vector of the present invention, transformant of the present invention, and antibody of the present invention. There can be provided a method of screening for compounds that inhibit or enhance a function of the polypeptide or protein of the present invention to regulate expression of the RB1 gene or protein by using at least one member of the group consisting of the polypeptide or protein of the present invention and the antibody of the present invention. For example, screening for the antagonist obtained by drug design based on the tertiary structure of the polypeptide, screening for an expression regulator at the genetic level that utilizes a protein expression system, screening for an antibody recognizing substance utilizing the antibody and the like can be utilized in a publicly known pharmaceutical screening system.

(Compound, Pharmaceutical Composition)

Compounds obtained by the above-described screening methods can be utilized as candidate compounds for the inhibitor, antagonist, activator or the like that regulates a function of the novel protein RB1CC1 and the polypeptide comprising the product derived therefrom to control expression of RB1 gene or protein. Compounds can also be utilized as candidate compounds for an inhibitor, antagonist, activator or the like for expression of the novel protein RB1CC1 and the polypeptide comprising the product derived therefrom at the genetic level. Examples of aforementioned candidate compounds for an inhibitor, antagonist, activator or the like include a protein, a polypeptide, a polypeptide without antigenicity, and a low molecular weight compound, and a low molecular weight compound is preferred.

Candidate compounds that were screened in the above manner can be selected in consideration of a balance between biological usefulness and toxicity to be prepared as pharmaceutical compositions to be used for treatment of osteosarcoma, leukemia or a tumor originating from the mammary gland, prostate gland, lung, or colon or the like. Further, the novel protein RB1CC1 comprising the present invention and the polypeptide comprising the product derived therefrom, nucleic acids encoding these and complementary strands thereof, vectors containing these base sequences, and antibodies that immunologically recognize the novel protein RB1CC1 and the polypeptide comprising the product derived therefrom can be used as pharmaceutical means, by themselves, that have an inhibitory, antagonizing or activating function with respect to interaction between the novel protein RB1CC1 and RB1 gene product and are used in treatment of breast cancer, prostrate cancer and the like . Here, the term “breast cancer, prostrate cancer and the like” includes a benign tumor and a malignant tumor, and in this connection, at the time of formulation, publicly known formulation means may be introduced in accordance with the substance for formulation, such as the polypeptide, protein, nucleic acid or antibody.

The novel protein RB1CC1 of the present invention and the polypeptide comprising the product derived therefrom, nucleic acids encoding these and complementary strands thereof, vectors containing these base sequences, and antibodies that immunologically recognize the novel protein RB1CC1 and the polypeptide comprising the product derived therefrom can be used as means for testing or diagnosing a disease with which expression of the polypeptide of the present invention or the activity thereof is related, such as a disease relating to expression of the novel protein RB1CC1 of the present invention or interaction with RB1 gene or the product thereof. In particular, they are useful as means for examination and diagnosis such as a diagnostic marker and/or reagent or the like for breast cancer, prostrate cancer and the like. Diagnosis can be conducted by utilizing interaction or reactivity with the nucleic acid sequence encoding the novel protein RB1CC1 to determine the abundance of a nucleic acid sequence of interest, and/or determine the biodistribution for the novel protein RB1CC1, and/or determine the abundance of the novel protein RB1CC1 in a test sample. More specifically, testing can be conducted utilizing the novel protein RB1CC1 as the diagnostic marker. As a method of determination, a publicly known antigen-antibody reaction system, enzyme reaction system, PCR reaction system or the like may be used. Further, a reagent kit or the like used in a method of examination and diagnosis is also included.

### EXAMPLES

The present invention is described in further detail hereunder on the basis of examples, however, the present invention is not limited by the following examples.

### Example 1

### cDNA of Human RB1CC1

In order to identify genes involved in MDR, we found a gene that expresses differentially in U-2 OS osteosarcoma cells and MDR-variant induced cells, to thereby identify a novel human gene. The gene was cloned using the set of primers (CC1-S1 and CC1-AS1) set forth in SEQ ID Nos: 5 and 26 and the set of primers (CC1-S2 and CC1-AS2) set forth in SEQ ID Nos: 6 and 25 in the sequence listing, and the nucleic acid sequence thereof was then determined using the primers set forth in SEQ ID Nos: 7 to 24. Further, the cDNA sequences at the 5′- and 3′-ends were identified using a commercially available rapid amplification kit for cDNA end sequences (RACE kit, manufactured by Roche) and the primers set forth in SEQ ID Nos: 27 to 37. The DNA and the amino acid sequence encoded thereby were analyzed using DNAs is Version 3.2 Sequence Analyzer (manufactured by Hitachi Software Engineering Co.) and PSORT II (http://www.yk.rim.or.jp/˜aisoai/molbio-j.html). Results showed that the cDNA had a length of 6.6 kb including an open reading frame (ORF) of 4782 nucleotides, encoding a protein comprising 1594 amino acids with a molecular weight of 180 kDa.

### Example 2

### cDNA of Mouse Rb1cc1

The mRNA of mouse muscle was employed as a template for amplification by RT-PCR, and cloning was then conducted using the set of primers (MCC1-S1 and MCC1-AS1) set forth in SEQ ID Nos: 53 and 73 and the set of primers (MCC1-S2 and MCC1-AS2) set forth in SEQ ID Nos: 54 and 72 in the sequence listing. The nucleic acid sequence was determined using primers set forth in SEQ ID Nos: 55 to 71 in the sequence listing. The cDNA of novel mouse protein Rb1cc1 was then identified using a similar method to Example 1, with the exception that rapid amplification of the cDNA was conducted using the primers (MCC-ASR1, MCC-ASR2, MCC-ASR3 and INTRON1ASR) set forth in SEQ ID Nos: 74 to 77 in the sequence listing as primers for the 5′-end RACE, and the primers (MCC-SR1, MCC-SR2, MCC3-S3, MCC3-S4, MCC3-AS2 and MCC3-AS3) set forth in SEQ ID Nos: 78 to 83 as primers for the 3′-end RACE. The cDNA encoding novel mouse protein Rb1cc1 has a strand length of 6518 bp including an open reading frame (ORF) of 4764 bp encoding 1588 amino acids. The gene of novel mouse protein Rb1cc1 had homology of 86% at the nucleic acid level and 89% at the protein level with the gene of novel human protein RB1CC1 (see SEQ ID Nos: 1 to 4).

### Example 3

### Analysis of MDR1 Gene and RB1CC1 Gene of the Present Invention

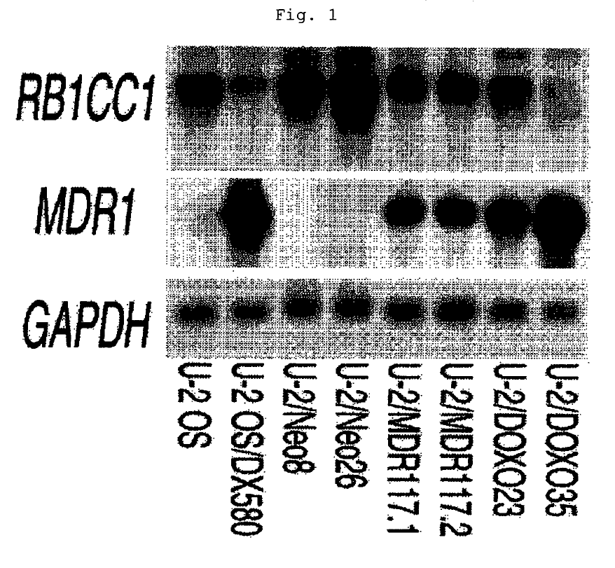

Expression levels of RB1CC1 gene and MDR1 gene in parental U2 OS cells and several kinds of MDR-variant cells were analyzed by Northern blotting. A probe hybridizing between nucleotide numbers 4190 and 4654 of the RB1CC1 gene sequence was used as a probe for analysis of RB1CC1 gene, and a probe hybridizing between nucleotide numbers 834 and 1119 of MDR1 gene was used for MDR1 gene. Probes were used after labeling with α-32P-dCTP in which phosphorus at an alpha position of deoxycytidine-3-phosphate was substituted with a radioactive isotope. Glyceraldehyde-3-phosphate dehydrogenase (GAPDH) was used as the indicator of mRNA expression. The results showed that the expression levels of both genes correlated inversely (FIG. 1).

### Example 4

### Preparation of Antibody and Western Blot Analysis

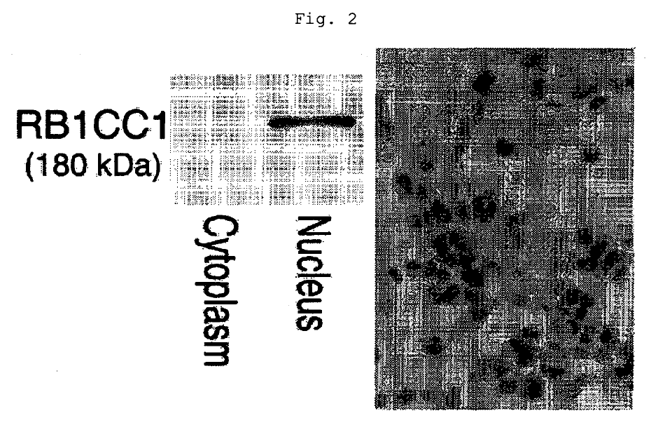

Three kinds of synthetic polypeptide were prepared which respectively comprised amino acids 642 to 658 (RB1CC-642), 744 to 757 (RB1CC-744) and 1104 to 1118 (RB1CC-1104) of the amino acid sequence of the novel protein RB1CC1 of the present invention. Rabbits were immunized by a conventional method with substances in which a cysteine residue had been introduced at the amino terminus of each polypeptide, and antibody was then obtained. After subjecting nuclear components and cytoplasmic components of U-2 OS cells to SDS-PAGE, respectively, analysis was carried out by Western blotting using the antibody prepared above. Results showed that RB1CC1 protein of a molecular weight of 180 kDa was present in the nucleus (FIG. 2).

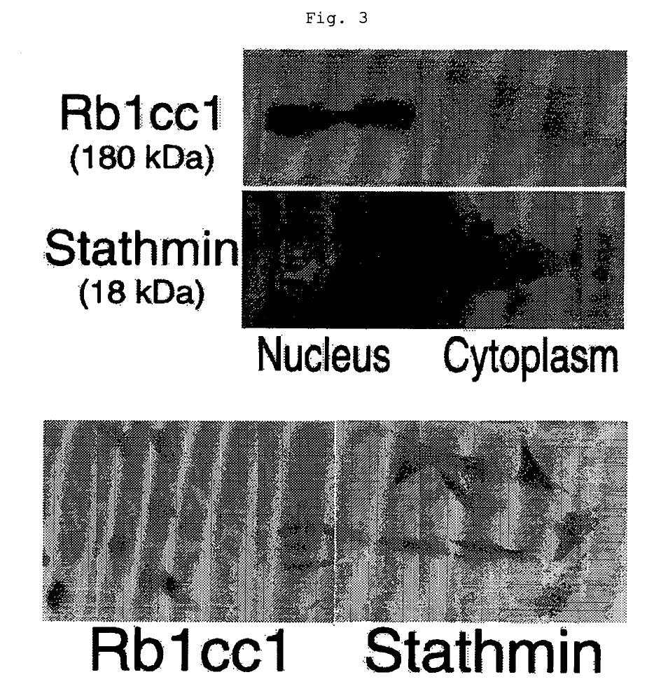

After subjecting nuclear components and cytoplasmic components of NIH3T3-3 cells of mouse to electrophoresis in a similar manner, Western blot analysis was conducted using the RB1CC-642 antibody. Detection of stathmin was simultaneously conducted using anti-stathmin rabbit antibody. Results showed that the Rb1cc1 protein is localized in the nucleus, while stathmin is present in cytoplasm. When same cells were subjected to immunocytochemical staining using each antibody and then compared, it was found that while the nucleus was stained with the RB1CC-642 antibody, the cytoplasm was stained with the anti-stathmin rabbit antibody (FIG. 3).

Above results showed that the novel protein RB1CC1 of the present invention is present in the nucleus of mammalian cells.

### Example 5

### Effect of Anticancer Agent on Expression of RB1CC1 Gene of the Present Invention

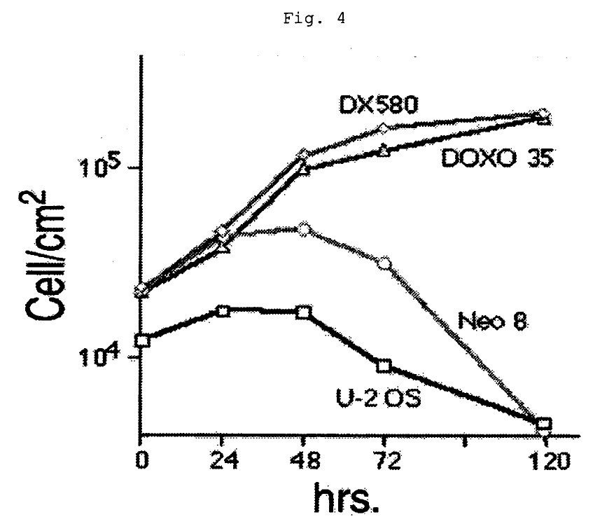

The influence of an anticancer agent was assessed for 4 kinds of cells that were treated with doxorubicin, including parent cells (U-2 OS), MDR variants of U-2 OS cells (U-2 OS/DX580) and U-2 OS cells introduced with the MDR1 gene (U-2/DOXO 35). The effect on cell proliferation in the presence of 450 ng/mL of the anticancer agent doxorubicin was examined. As shown in FIG. 4, results indicated that while cell proliferation was suppressed by the anticancer agent in parental U2 OS cells and control cells introduced with a gene (U-2/Neo8), the anticancer agent had no effect on MDR variants of U-2 OS cells (U-2 OS/DX580) and U-2 OS cells introduced with MDR1 gene (U-2/DOXO 35) and cell proliferation continued for 120 hours or more (FIG. 4).

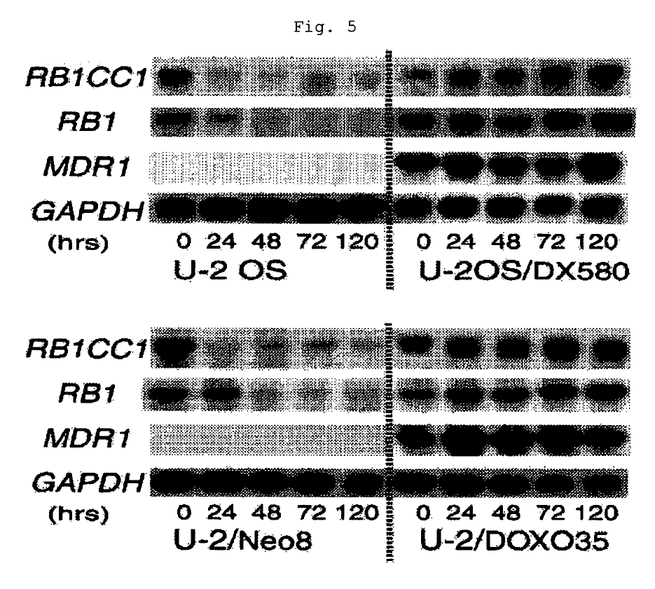

mRNA expression levels of cells that were obtained over time in the above-mentioned experiment were analyzed. Analysis was conducted for the novel gene RB1CC1 gene of the present invention, the RB1 gene and the MDR1 gene, respectively, in the same manner as Example 3 with the exception that expression levels of the RB1 gene were detected using a probe hybridizing to the site at nucleotides 336 to 675 of the nucleotide sequence of human RB1 mRNA. Results are shown in FIG. 5. For parental U2 OS cells and control cells introduced with a gene (U-2/Neo8) for which the effect of the anticancer agent was observed, expression of the RB1CC1 gene decreased over time. In contrast, in MDR variants of U-2 OS cells (U-2 OS/DX580) and U-2 OS cells introduced with MDR1 gene (U-2/DOXO 35), expression level of RB1CC1 gene was not inhibited by treatment with doxorubicin, and expression of RB1CC1 gene increased. In these cells, RB1CC1 gene expression and RB1 gene expression correlated (FIG. 5).

### Example 6

### Expression of RB1 Gene and RB1CC1 Gene of the Present Invention

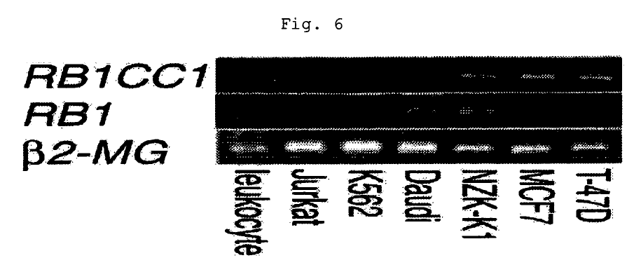

The expression of RB1CC1 gene and RB1 gene in various cancer cells was assessed by semi-quantitative RT-PCR. Cell lines used were SARG, IOR/OS 9, 10, 14, 15, 18, MOS (these were obtained from surgical samples of advanced human osteosarcoma), Saos-2, HOS, MCF-7, T-47D, BT-20, SK-BR3, ZR75-1, MDA-MB-231, Daudi, Jurkat and K562 (these were purchased from the American Type Culture Collection), NZK-K1 (this was established from breast cancer tissue of a 46-year old female), LK2, QG56, EBC1 and SBC2 (these were provided by Doctor Tatsuhiko Narita of Aichi Cancer Center). 2 μg of RNA was extracted from each cell line, and subjected to 22 to 30 cycles of RT-PCR for amplification. Publicly known primers were synthesized and used as primers for the RB1 gene (Sauerbrey et al., 1996). The combination of primers set forth in SEQ ID Nos: 19 and 20 in the sequence listing (CC1-S and CC1-AS) were used as primers for amplification of RB1CC1. β₂-microglobulin was used as a control. In all of these cells, expression of RB1CC1 gene correlated closely with that of RB1 gene. FIG. 6 shows results for one case of normal leukocyte and six cancer cells: T-47D, MCF7, NZK-K1, Daudi, K562 and Jurkat (FIG. 6).

### Example 7

### Expression of RB1CC1 Gene and RB1 Gene of the Present Invention in Organs

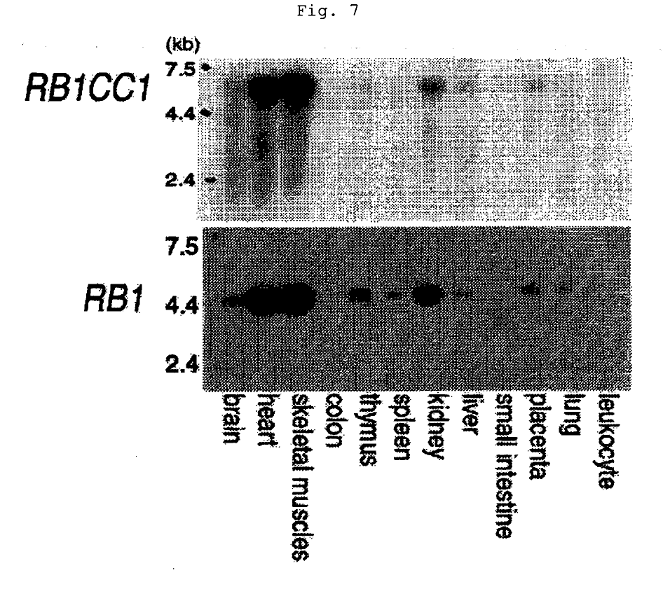

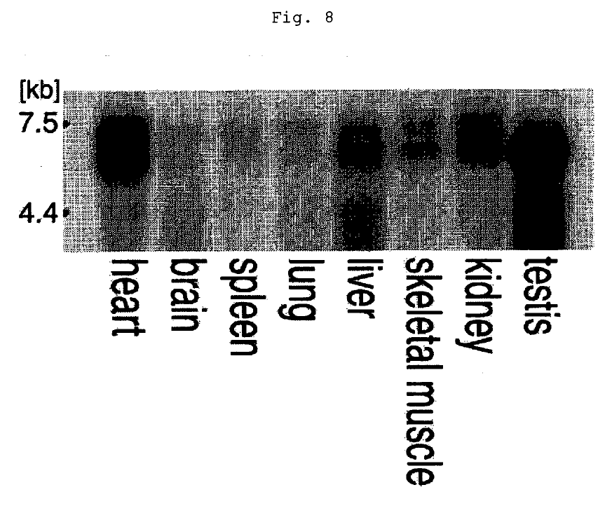

Northern blot analysis was conducted for RB1CC1 gene and RB1 gene expressing in nonneoplastic tissue of human brain, heart, skeletal muscle, colon, thymus, spleen, kidney, liver, small intestine, placenta, lung and leukocyte, respectively, using commercially available MTN Blots (manufactured by Clontech). Results are shown in FIG. 7. Both genes were expressed strongly in heart and skeletal muscle, while expression was weak in colon, small intestine, lung and leukocyte. However, expression of RB1CC1 gene and RB1 gene correlated. Northern blot analysis was also conducted for Rb1cc1 gene expressing in respective tissues of heart, brain, spleen, lung, liver, skeletal muscle, kidney and testis of mouse. Results are shown in FIG. 8. Transcription products of 6.2 kb and 6.8 kb were expressed strongly in heart, while expression was observed to a certain extent in kidney, liver and skeletal muscle. The principal expression in testis was 6.2 kb, while expression was weak in lung and spleen (FIG. 7, FIG. 8).

### Example 8

### Expression of RB1 Gene Induced by Introduction of RB1CC1 Gene of the Present Invention

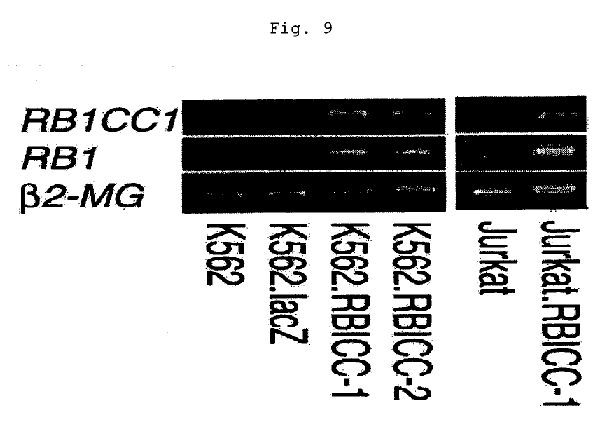

Jurkat and K562 cells that had weak expression levels for both RB1CC1 gene and RB1 gene among cells shown in Example 6 were subjected to exogenous introduction of RB1CC1 gene to examine changes in the expression of RB1 gene. A 4.9-kb gene that included the complete coding region of the RB1CC1 molecule was incorporated into pCR3.1-Uni vector (manufactured by Invitrogen), which was then cloned to prepare an RB1CC1 expression vector (pCR-RB1CC). The thus-prepared expression vector was incorporated into K562 and Jurkat cells to prepare RB1CC1 transformed cells. A control was prepared by incorporating lac Z gene into pCR3.1-Uni vector. Respective expression levels of RB1CC1 gene and RB1 gene in parent cells and transformed cells (cells introduced with RB1CC1 gene) were examined in a similar manner to Example 6. FIG. 9 shows the results. Although expression of both RB1CC1 gene and RB1 gene was weak in untransformed cells and cells into which the lac Z gene was incorporated, it was found that in cells incorporated with RB1CC1 gene, the RB1CC1 gene expression was strong as expected and the RB1 gene was also strongly expressed, showing that expression of the RB1 gene was also induced by introduction (exogenous expression) of the RB1CC1 gene (FIG. 9).

### Example 9

### RB1 Gene Promoter Transcriptional Activity of RB1CC1 Gene of the Present Invention

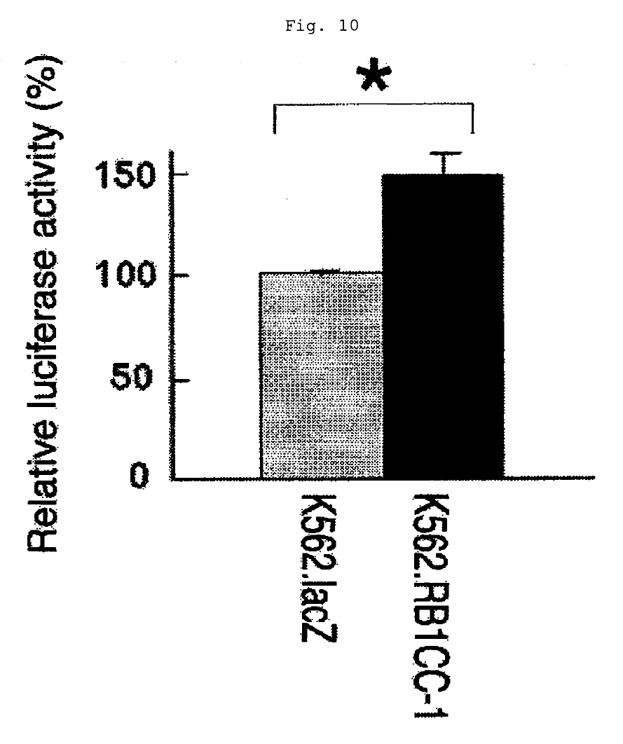

We examined whether introduction of the RB1CC1 gene enhanced the transcriptional activity of the promoter region of RB1 gene. A gene of RB1 promoter region of approximately 2 kb was amplified with the pair of primers 5′-GAA GAT CTT TGA AAT TCC TCC TGC ACC A-3′ (Bg1.RbPro-S) and 5′-CCC AAG CTT AGC CAG CGA GCT GTG GAG-3′ (Hind.RbPro-AS), and incorporated into PicaGene Basic vector 2 (manufactured by Toyo Ink Mfg.Co., Ltd.). Then, RB1 promoter which controls expression of firefly luciferase was used to prepare pGV-RbPro vector. The prepared pGV-RbPro vector was then retranscribed with pRL-SV40 encoding the sea pansy luciferase gene, as an internal control, and incorporated into K562 cell using LIPOFECTAMINE PLUS reagent (manufactured by GIBCO-BRL). Results of analysis conducted after 48 hours using a double luciferase assay system (Toyo Ink Mfg. Co., Ltd.) showed that K562 cell introduced with RB1CC1 gene exhibited strong luciferase activity compared to K562 cell incorporated with lac Z as a control, showing that introduction of the RB1CC1 gene enhanced the transcriptional activity of RB1 gene promoter (FIG. 10).

### Example 10

### Loss of Heterozygosity at Locus (D8S567) of RB1CC1 Gene in Primary Breast Cancer

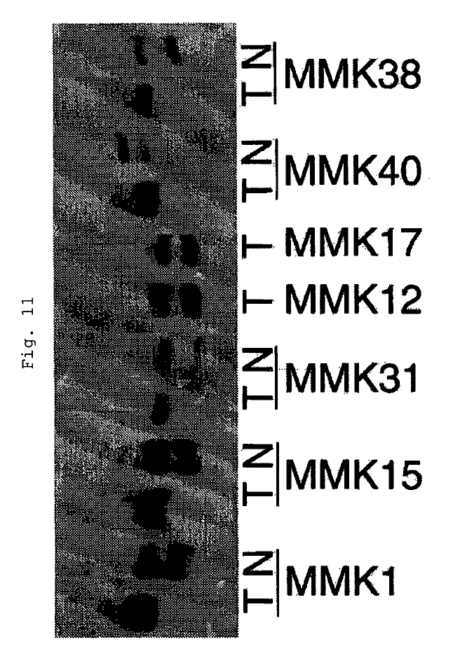

DNA samples of cancer tissue and genome DNA from same patients were amplified by PCR and the amplification products were analyzed using 8% urea-denatured polyacrylamide gel electrophoresis. Results obtained by silver staining after electrophoresis are shown in FIG. 11. While two bands were observed for the genome DNA of each patient to indicate retention of heterozygosity, only one band was detected in five cases of DNA of cancer tissue, indicating loss of heterozygosity (FIG. 11).

### Example 11

### Analysis of Mutation of RB1CC1 Gene of the Present Invention in Breast Cancer

Mutations of RB1CC1 gene were identified by analyzing the genetic sequence of cDNA samples that were amplified using ELONGASE System (manufactured by GIBCO-BRL) with the pair of primers (CC1-S2 and CC1-AS2) set forth in SEQ ID Nos: 6 and 25 that were used in Example 1, using ABI PRISM 310 genetic analyzer and the primers set forth in SEQ ID Nos: 7 to 24 in the sequence listing. As a result, 7 cases of mutation were verified among 35 cases of breast cancer, and 9 kinds of variants were verified. This result was reconfirmed using primers set forth in SEQ ID Nos: 38 to 52. Results are shown in Table 2.

TABLE 2
Mutations of RB1CC1 gene in primary breast cancer

| sample | nucleotide | location | predicted | genome | State of RB1CC1 gene | | State of RB1 | |
|---|---|---|---|---|---|---|---|---|
| name | mutation | (exon) | influence | DNA | allele | protein | LOH | protein |
| MMK3 | c.11_4800del | 3-24 | Y4fsX4 | wild type | plural heterozygous deletions | (−) | (−) | ↓ ↓ |
| | c.325_1585del | 5-11 | P109fsX122 | wild type | plural heterozygous deletions | (−) | (−) | |
| MMK6 | c.10_4798del | 3-24 | Y41sX48 | wild type | plural heterozygous deletions | (−) | (−) | (−) |
| | c.1233_4633del | 9-23 | D411fsX431 | wild type | plural heterozygous deletions | (−) | (−) | |
| MMK1 | c.957_4785del | 7-24 | N319fsX368 | wild type | plural heterozygous deletions | (−) | (−) | ↓ ↓ |
| MMK15 | c.1635_4719del | 12-24 | S545fsX557 | wild type | plural heterozygous deletions | (−) | (−) | (−) |
| MML31 | c.212_4188del | 5-24 | 171fsX111 | wild type | plural heterozygous deletions | (−) | (−) | (−) |
| MMK38 | c.241_4621del | 5-22 | O81fsX99 | wild type | plural heterozygous deletions | (−) | (−) | ↓ ↓ |
| MMK40 | c.591_4678del | 7-23 | S197fsX212 | wild type | plural heterozygous deletions | (−) | (−) | ↓ ↓ |

(−): absent,
↓ ↓: significantly decreased
LOH: loss of heterozygosky

### Example 12

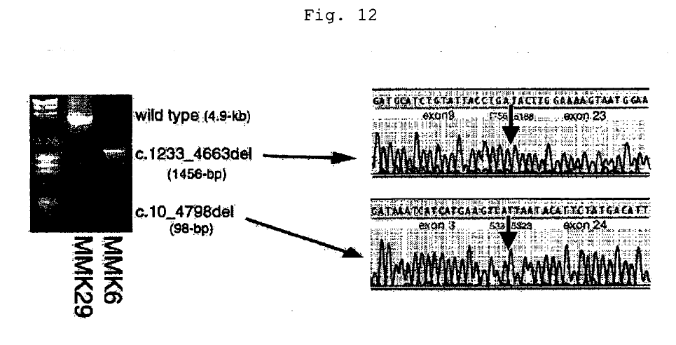

FIG. 12 shows results of analysis of PCR products for MMK6 in which mutation was observed in RB1CC1 gene and MMK29 in which mutation was not observed among samples analyzed in Example 11, as well as the results of-genetic sequence analysis corresponding thereto. It was found that a gene of 4.9 kb expressed in MMK29 that was without mutation, while the 4.9-kb expression was not observed in MMK6 with mutation and expression of gene fragments (1456 bp and 98 bp) was observed (FIG. 12).

### Example 13

### Analysis by Western Blotting

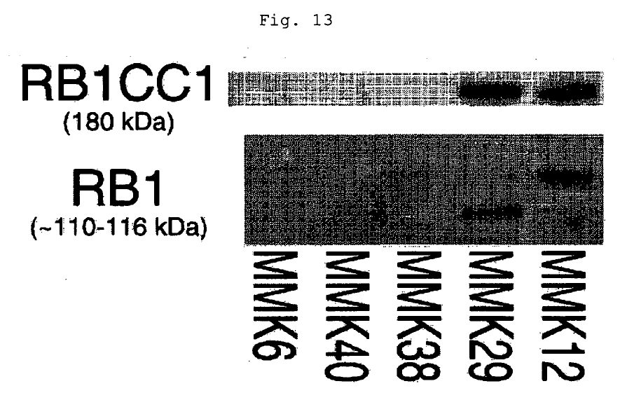

From the samples analyzed in Example 11, expression of the novel protein RB1CC1 and the RB1 protein was verified by Western blotting in 3 cancers (MMK6, MMK40, MMK38) in which mutation was observed in RB1CC1 gene and 2 cancers (MMK12, MMK29) in which mutation was not observed. After subjecting extracted protein to 5% SDS-polyacrylamide gel electrophoresis, and then transferring to PVDF membrane, reaction was conducted with the anti-human RB1CC1 antiserum (α-RB1CC-642) prepared in Example 4. The RB1 protein was reacted with RB1 monoclonal antibody (G3-245, manufactured by PharMingen Inc.). After reaction, detection was carried out using ECL reagent (manufactured by Amersham Biosciences) The results are shown in FIG. 13. While novel protein RB1CC1 having a molecular weight of 180 kDa and RB1 protein of a molecular weight of 110 to 116 kDa both expressed in MMK12 and MMK29 without mutation, in contrast, expression of either protein was not observed in any of 3 cancers with a mutation (FIG. 13).

### Example 14

### Immunohistological Staining

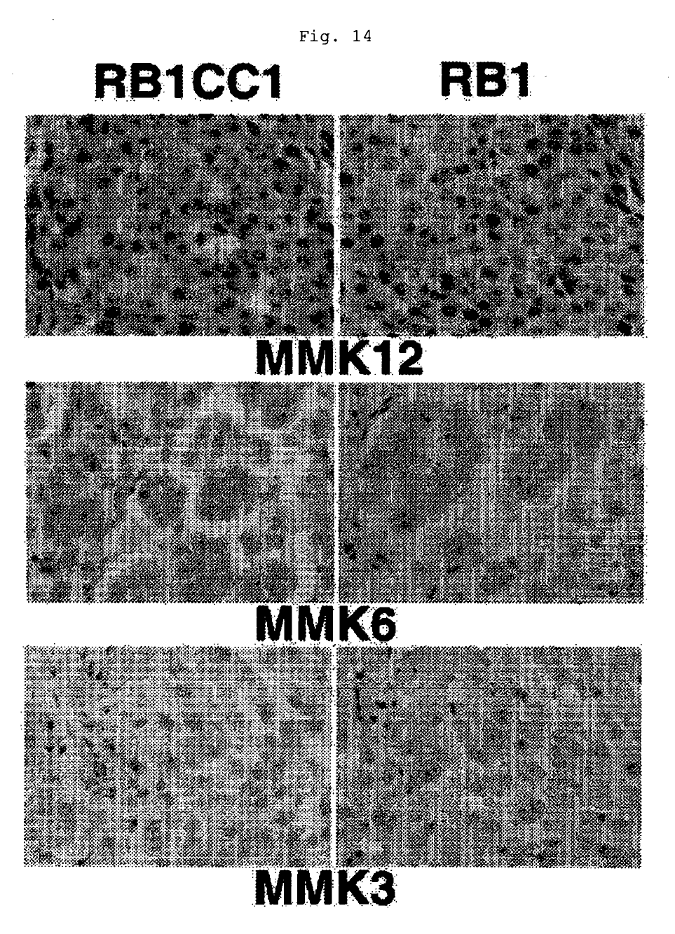

Immunohistological staining was conducted for 2 cancers (MMK3, MMK6) in which mutation in RB1CC1 gene was observed and 1 cancer (MMK 12) in which mutation was not observed among samples analyzed in Example 11. The antibody used for reaction was the same as that in Example 13, and the antibody was reacted with tissue sections prepared from paraffin blocks obtained from each of cancer samples. As shown in FIG. 14, the expression levels of novel protein RB1CC1 and RB1 protein correlated, and it was verified that expression levels were clearly lower in 2 cancers (MMK3, MMK6) in which mutation in RB1CC1 gene was observed compared to the cancer (MMK 12) in which mutation was not observed (FIG. 14).

### Example 15

54 samples of primary breast cancer tissue were assayed by immunohistological staining in a similar manner to Example 14, and the RB1CC1 protein was not detected in 8 samples (corresponding to 15%). Then, RB1 protein expression was absent or significantly lowered in all of the samples.

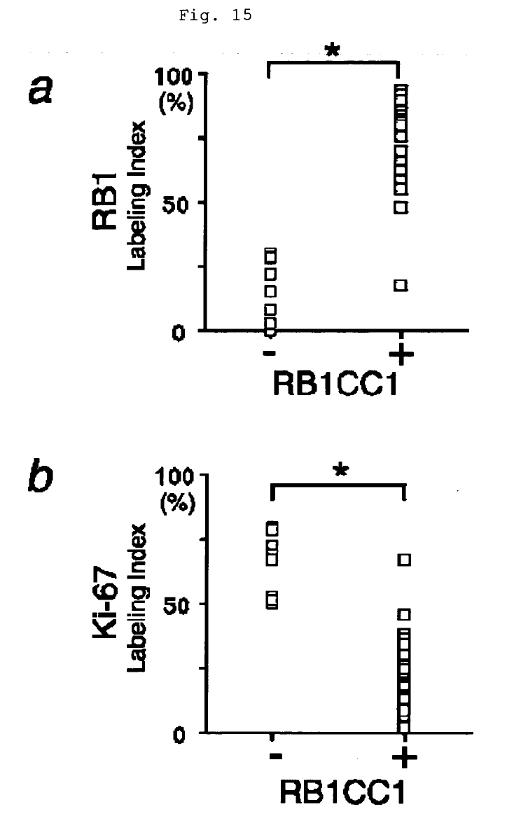

For 46 cases expressing RB1CC1 protein, the RB1 protein was simultaneously expressed in 45 cases. When the RB1 protein expression was compared with the RB1CC1 positive group and negative group by stain indication using immunohistological staining (indication showing as a percentage the ratio of the number of cells stained among 1000 or more cells), the RB1CC1 positive group and negative group were found to show a positive correlation with RB1CC1 expression, with 78.6±13.9% and 13.6±12.1%, respectively (FIG. 15a). Meanwhile, when immunohistological staining for Ki-67 was conducted using mouse monoclonal antibody (NCL-Ki-67-MMI, manufactured by Novocastra Inc.), the stain indication was 20.3±12.8% for the RB1CC1 positive group and 65.0 ±12.2% for the negative group, showing a clearly inverse correlation with RB1CC1 expression (FIG. 15b).

These results indicate that in cancers in which expression of RB1CC1 protein is suppressed, the cell proliferation marker Ki-67 is expressed in large amounts, and proliferation of cancer cells flourishes. It was thus found that assaying using a combination of RB1CC1 protein and Ki-67 is useful for cancer diagnosis.

By testing for the novel gene (RB1CC1 gene) of the present invention and the protein (RB1CC1) thereof, information that is useful for the diagnosis of cancer cell proliferation and cancer can be provided.

1. A protein or polypeptide which is present in nucleus of human or animal cell and which has a transcription factor function and/or a function that can induce expression of retinoblastoma gene (RB1 gene) or a gene product thereof.

2. The human protein according to claim 1, which is a polypeptide or protein selected from a group consisting of: (1) a polypeptide or protein represented by an amino acid sequence set forth in SEQ ID No: 1 in the sequence listing; (2) a polypeptide containing an amino acid sequence comprising at least five amino acids of the amino acid sequence of the polypeptide or protein; (3) a polypeptide or protein having homology of at least approximately 70% at the amino acid sequence level with the polypeptide or protein; and (4) a protein or polypeptide having a mutation or induced mutation such as a deletion, substitution or addition of one to several amino acids relative to the amino acid sequence of the polypeptide or protein according to any one of the preceding (1) to (3).

3. The animal protein according to claim 1 that is a protein derived from mouse, and which is a polypeptide or protein selected from the group consisting of: (1) a polypeptide or protein represented by an amino acid sequence set forth in SEQ ID No: 2 in the sequence listing; (2) a polypeptide containing an amino acid sequence comprising at least five amino acids of the amino acid sequence of the polypeptide or protein; (3) a polypeptide or protein having homology of at least approximately 70% at the amino acid sequence level with the polypeptide or protein; and (4) a protein or polypeptide having a mutation or induced mutation such as a deletion, substitution or addition of one to several amino acids relative to the amino acid sequence of the polypeptide or protein according to any one of the preceding (1) to (3).

4. A nucleic acid coding for the polypeptide or protein according to claim 1, or a complementary strand thereof.

5. A nucleic acid hybridizing under stringent conditions with the nucleic acid according to claim 3 or the complementary strand thereof.

6. A nucleic acid represented by a base sequence comprising at least 15 consecutive bases of the base sequence of a nucleic acid set forth in SEQ ID Nos: 3 to 4 in the sequence listing or a complementary strand thereof, wherein a polypeptide expressed by transcription of the nucleic acid is the polypeptide according to claim 1.

7. A recombinant vector containing the nucleic acid according to claim 4.

8. A transformant that was transformed with the recombinant vector according to claim 7.

9. A method for producing the polypeptide or protein according to claim 1, comprising a step of culturing the transformant with the recombinant vector containing nucleic acid coding for the polypeptide or protein.

10. Nucleic acid primers set forth in SEQ ID Nos: 5 to 132 in the sequence listing, which hybridize under stringent conditions with the nucleic acid according to claim 4 or the complementary strand thereof.

11. An antibody that immunologically recognizes the polypeptide or protein according to claim 1.

12. A method of screening for compounds that inhibit or enhance a function that can induce transcription factor activity and/or expression of RB1 gene of the polypeptide or protein according to claim 1, wherein the method utilizes the polypeptide, the protein, or an antibody that immunologically recognizes the polypeptide or protein.

13. A method of screening for compounds that interact with the nucleic acid according to claim 4 to inhibit or enhance expression of the nucleic acid, wherein the method utilizes the nucleic acid, a recombinant vector containing the nuclic acid a transformant that was transformed with the recombinant vector, or nucleic acid primers set forth in SEQ ID NOS: 5 to 132 in the sequence listing which hybridize under stringent conditions with the nucleic acid.

14. A compound that was screened by the screening method according to claim 12.

15. A compound that inhibits or enhances transcription factor activity and/or a function that can induce expression of RB1 gene of the polypeptide or protein according to claim 1.

16. A compound that interacts with the nucleic acid according to claim 4 to inhibit or enhance expression of the nucleic acid.

17. A pharmaceutical composition for use in treatment of multidrug resistance that is resistance to treatment with anticancer agents, wherein the pharmaceutical composition comprises the polypeptide or protein according to claim 1, a nucleic acid coding for the polypeptide or protein or a complementary strand thereof, a recombinant vector containing the nucleic acid, a transformant that was transformed with the recombinant vector, nucleic acid primers set forth in SEQ ID NOS: 5 to 132 in the sequence listing which hybridize under stringent conditions with the nucleic acid, an antibody that immunologically recognizes the polypeptide or protein, or a compound that interacts with nucleic acid to inhibit or enhance expression of the nucleic acid.

18. A method of testing and diagnosing a disease related with expression or activity of the polypeptide or protein according to claim 1, wherein the method comprises a step of conducting analysis employing (a) a nucleic acid encoding the polypeptide or protein and/or (b) the polypeptide or protein, as a marker in a sample.

19. The method of testing and diagnosing according to claim 18, which is a method of testing cancer cells or a method for diagnosing a cancer.

20. The method according to claim 18 which detects expression, increase, decrease, lack or the like of all or a part of the polypeptide or protein, wherein the method utilizes an antibody that immunologically recognizes the polypepetide.

21. The method according to claim 18 which detects expression, mutation, lack or insertion or the like of all or a part of a gene encoding the polypeptide or protein through a step of amplifying a gene encoding the polypeptide or protein utilizing at least one of nucleic acid primers set forth in SEQ ID NOS: 5 to 132 in the sequence listing, which hybridize under stringent conditions with the nucleic acid.

22. The method according to claim 18, wherein the method combines assay of expression, increase, decrease, mutation, lack or insertion or the like of all or a part of tumor-suppressor gene retinoblastoma gene (RB1 gene) or the gene product thereof (RB1 protein).

23. The method according to claim 18, wherein the method combines assay of expression, increase, decrease, mutation, lack or insertion or the like of all or a part of multidrug resistance gene (MDR1 gene) or the gene product thereof (MDR1 protein: P-glycoprotein).

24. The method according to claim 18, wherein the method combines assay of expression, increase, or decrease or the like of all or a part of the cell proliferation marker, Ki-67 protein.

25. A method that tests drug sensitivity of a cancer cell using the method according to claim 23.

26. A kit and a reagent for assay or diagnosis, for use in the method according to claim 18.
